(19) United States
(12) Patent Application Publication
(10) Pub. No.: US 2012/0135661 A1
(43) Pub. Date: May 31, 2012

(54) SOLVENT FOR MAKING LIQUID CRYSTAL ALIGNMENT FILM, MATERIALS FOR THE ALIGNMENT FILM, AND METHOD FOR MANUFACTURING LIQUID CRYSTAL DISPLAY

(75) Inventors: Yasuo IMANISHI, Katano (JP); Hidehiro Sonoda, Mobara (JP); Noboru Kunimatsu, Chiba (JP); Yasushi Tomioka, Mobara (JP); Masaki Matsumori, Tokai (JP)

(73) Assignee: Hitachi Displays, Ltd.

(21) Appl. No.: 13/301,819

(22) Filed: Nov. 22, 2011

(30) Foreign Application Priority Data
Nov. 26, 2010 (JP) 2010-263429

(51) Int. Cl. H01J 9/00; C09K 3/00; C07C 49/76

(52) U.S. Cl. 445/24; 568/335; 252/182.28

(57) ABSTRACT

Provided is a solvent for forming an alignment film capable of fabricating a liquid crystal alignment film having high quality, an improved imidization ratio, large surface anisotropy, and a strong anchoring force. The solvent for forming an alignment film including a polymer for a liquid crystal display includes the polymer of the alignment film including polyimide and a variable compound capable of changing a chemical structure for evaporating after imidizing and sintering, which is in a liquid state during a film-forming process of coating, imidizing, and sintering polyamic acid which is a precursor of polyimide.

### CLAIM OF PRIORITY

The present application claims of priority from Japanese patent application JP 2010-263429 filed on Nov. 26, 2010, the content of which is hereby incorporated by reference into this application.

### BACKGROUND OF THE INVENTION

1. Field of the Invention

The present invention relates to a solvent for making an alignment film which is used when a liquid crystal display having a high contrast and a low afterimage characteristic is manufactured.

2. Description of the Related Art

A liquid crystal display has an expanded use due to characteristics such as high display quality, a thin form, a light weight, low consumption power, and the like and is used for various uses, such as portable monitors such as a monitor for a portable phone, a monitor for a digital still camera, and the like, a monitor for a desktop personal computer, a monitor suitable for printing or design, a medical monitor, a liquid crystal television, and the like. According to expansion of the uses, the liquid crystal display is required to have more high-definition and high-quality and particularly, is strongly required to have high-luminance due to high-transmittance and low consumption power. Further, according to prevalence of the liquid crystal display, the liquid crystal display is strongly required to have low costs.

In general, the liquid crystal display displays an image by applying an electric field to liquid crystal molecules of a liquid crystal layer interposed between a pair of substrates to change an alignment direction of the liquid crystal molecules and change an optical characteristic of the liquid crystal layer generated therethrough. When the electric field is not applied, the alignment direction of the liquid crystal molecules is defined by an alignment film in which a rubbing process is preformed to a surface of a polyimide thin film. In the related art, an active-driving liquid crystal display including a switching element such as a thin film transistor (TFT) every pixel displays an image by including an electrode on a pair of substrates with the liquid crystal layer interposed therebetween, setting a direction of the electric field applied to the liquid crystal layer so as to be substantially perpendicular to the substrate surface, so-called, a vertical switching mode, and using optical activity of light of the liquid crystal molecules configuring the liquid crystal layer. As a representative liquid crystal display of the vertical switching mode, a twisted nematic (TN) mode or a vertical alignment (VA) mode is known. There is one problem in that the TN mode or VA mode liquid crystal display has a narrow viewing angle. Accordingly, as a display mode achieving a wide viewing angle, an in-plane switching (IPS) mode or a fringe-field switching (FFS) mode is known. The IPS mode and the FFS mode which are a so-called in-plane switching display mode, in which a comb-shaped electrode is formed at one side of a pair of the substrates and the generated electric field has a component substantially parallel to the corresponding substrate surface, rotate the liquid crystal molecules configuring the liquid crystal layer in the plane substantially parallel to the substrate, thereby displaying an image by using birefringence of the liquid crystal layer. The IPS mode and the FFS mode have advantages of having a wide viewing angle and a low-load capacity by the in-plane switching of the liquid crystal molecules as compared with the known TN mode and the modes are full of promise as a new liquid crystal display replacing the TN mode, such that recently, the modes have rapidly developed.

Liquid crystal display elements control alignment states of the liquid crystal molecules in the liquid crystal layer according to an electric field or not. That is, upper and lower polarizers installed outside the liquid crystal layer is in a completely perpendicular state to each other to generate a phase difference by an alignment state of the middle liquid crystal molecules, thereby forming a contrast state. In order to control the alignment state in the state where the electric field is not applied to the liquid crystal, a polymer thin film called an alignment film is formed on the substrate surface and the liquid crystal molecules are arranged by an intermolecular interaction of van der Waals' force between polymer chains and the liquid crystal molecules at an interface in an arranged direction of the polymers. The interaction is also called an alignment regulatory force, liquid crystal alignment ability, or an alignment processing. In many cases, polyimide is used in the alignment film of the liquid crystal display. As a forming method thereof, a solvent is removed by dissolving polyamic acid as a precursor of polyimide in various solvents to coat the dissolved polyamic acid solution on the substrate by a spin coating or printing method and heating the substrate at a high temperature of 200° C. or more and simultaneously, polyamic acid reacts with polyimide by an imidized-cyclization reaction. In this case, a thickness of the film which is a thin film is about 100 nm. Polyimide polymer chains on the surface are aligned in a predetermined direction by rubbing the surface of the polyimide thin film by a rubbing cloth in the direction, thereby implementing high anisotropy of the surface polymer. However, there are problems of generation of static electricity or impurities due to the rubbing, ununiformity of the rubbing due to unevenness of the substrate surface, and the like. Accordingly, a photoalignment method which controls a molecular alignment by using polarized light without requiring a contact with the rubbing cloth is adopted.

The photoalignment method of the liquid crystal alignment film includes a photoisomerization type in which a geometric arrangement in molecules is changed by irradiation a polarized ultraviolet light such as an azo pigment, a photodimerization type in which molecular backbones of cinnamate, coumalin, chalcone, and the like are chemically bonded with each other by the polarized ultraviolet light, and the like. However, a photolytic type, in which only the polymer chain arranged in a direction is cut and cleaved by irradiating the polarized light to the polymer and the polymer chain in a direction perpendicular to the polarized direction remains, is suitable for the photoalignment of polyimide which is reliable and available as the liquid crystal alignment film. An initial principle of the photoalignment method is disclosed in “Nematic Homogeneous Alignment by Photo Depolymerization of Polyimide” (HASEGAWA Masaki, TAIRA Yoichi, The 20th Japanese Liquid Crystal Conference Papers, 232 to 233 pages, in 1994). The method has been considered in various liquid crystal display modes, but among them, the IPS mode is disclosed in Japanese Patent Application Laid-Open Publication No. 2004-206091 as a liquid crystal display capable of reducing a display defect due to a change in an initial alignment direction and having a stable liquid crystal alignment, productivity, and a high-quality image having an improved contrast ratio. The alignment control ability is given by an alignment processing performing a secondary processing of at least one of heating, infrared-irradiation, far-infrared-irradiation, electron-beam irradiation, and radiation in polyamic acid or polyimide configured by cyclobutane tetracarboxylic acid dianhydride and/or a derivative thereof, and aromatic diamine. In addition, particularly, since at least one processing of the heating, the infrared-irradiation, the far-infrared-irradiation, the electron-beam irradiation, and the radiation is performed together with the polarized light irradiation processing at the same time, the present invention is more effective and since the imidized-sintering processing of the alignment control film is performed together with the polarized light irradiation processing at the same time, the present invention is more effective. In particular, when at least one processing of the heating, the infrared-irradiation, the far-infrared-irradiation, the electron-beam irradiation, and the radiation is performed on the liquid crystal alignment film in addition to the polarized light irradiation, a temperature of the alignment control film is in the range of 100° C. to 400° C. and preferably, 150° C. to 300° C. In addition, the heating, the infrared-irradiation, and the far-infrared-irradiation may be performed together with the imidized-sintering of the alignment control film, such that the present invention is effective.

### BRIEF SUMMARY OF THE INVENTION

In the related art, the alignment film is formed by dissolving polyamic acid which is a precursor of polyimide in a high-polarity solvent, and then, coating, and drying the dissolved polyamic acid to form the thin film, and then, performing an imidization reaction by heating. However, as the imidization progresses, the linearity of the polymer chain increases, but viscosity also increases at the same time, such that the imidization at a predetermined degree or more did not progress. As a result, since polyimide including a partially bent portion is formed, anisotropy of the molecular backbone itself giving the surface anisotropy is deteriorated, such that it was difficult to give the high anchoring force.

Further, when the imidization reaction is performed in a dilute solution in advance, and then, the coating, the drying, and the forming of the thin film is performed, the solubility in the solvent is deteriorated due to the high imidization, such that it is difficult to form a desired thin film.

The present invention has been made in an effort to provide a solvent for forming an alignment film capable of fabricating a liquid crystal alignment film having high quality, an improved imidization ratio, large surface anisotropy, and a strong anchoring force.

According to an embodiment of the present invention, there is provided a solvent for forming an alignment film including a polymer for a liquid crystal display including: a variable compound capable of changing a chemical structure during a film-forming process of coating the polymer and forming the alignment film.

In addition, the solvent for forming an alignment film may include the polymer of the alignment film including polyimide and a variable compound which is in a liquid state during a film-forming process of coating, imidizing, and sintering polyamic acid which is a precursor of polyimide, but changes a chemical structure for evaporating after imidizing and sintering.

According to another embodiment of the present invention, there is provided an alignment material including: the solvent for forming an alignment film and polyamic acid which is a precursor of polyimide.

According to yet another embodiment of the present invention, there is provided a method of manufacturing a liquid crystal display as a process of forming an alignment film including a polymer for a liquid crystal display, including: coating on a substrate a solution in which a polymer is dissolved in the solvent for forming an alignment film; imidizing and sintering the coated polymer; changing a chemical structure of the variable compound included in the solvent for forming an alignment film; removing the solvent having the changed chemical structure; and irradiating a polarized light for photoalignment on the remaining polymer thin film.

According to the embodiments of the present invention, quality of a liquid crystal alignment film, for example, an imidization ratio of polyimide is improved, linearity of a polymer chain is improved, strong surface anisotropy and anchoring force may be acquired in photoalignment processing, and a glass transition temperature of the acquired polyimide is improved, such that a dynamic strength increases and an afterimage of the liquid crystal display can be prevented.

### BRIEF DESCRIPTION OF THE DRAWINGS

FIG. 1A is a schematic diagram illustrating a solution state of a polyamic acid solution;

FIG. 1B is a block diagram illustrating a process of forming a light alignment film of polyimide by using a solvent for forming an alignment film according to an embodiment of the present invention;

FIG. 2A is an explanatory diagram illustrating a structural change in a process of forming materials for a polyimide alignment film including a variable compound according to an embodiment of the present invention;

FIG. 2B is an explanatory diagram illustrating a structural change in a process of forming materials for a polyimide alignment film including a variable compound according to an embodiment of the present invention;

FIG. 2C is an explanatory diagram illustrating a structural change in a process of forming materials for a polyimide alignment film including a variable compound according to an embodiment of the present invention;

FIG. 3A is an explanatory diagram illustrating in-plane anisotropy of a thin film of materials for a polyimide alignment film including a variable compound according to an embodiment of the present invention;

FIG. 3B is a diagram illustrating wavelength dependence of absorbance with respect to an alignment film which is subject to a light alignment processing;

FIG. 4A is an explanatory diagram illustrating a surface anisotropy meter of a thin film of materials for a polyimide alignment film including a variable compound according to an embodiment of the present invention;

FIG. 4B is a schematic diagram illustrating square root of a photoelectron detection amount and excited energy of a spectroscopic light source;

FIG. 5 is an explanatory diagram illustrating an anchoring force meter of a liquid crystal cell using a thin film of materials for a polyimide alignment film including a variable compound according to an embodiment of the present invention;

FIG. 6A is a schematic block diagram illustrating an example of a schematic configuration of a liquid crystal display according to an embodiment of the present invention.

FIG. 6B is a schematic circuit diagram illustrating an example of a circuit configuration of one pixel of a liquid crystal panel;

FIG. 6C is a schematic plan view illustrating an example of a schematic configuration of a liquid crystal panel;

FIG. 6D is a schematic cross-sectional view illustrating an example of a cross-sectional configuration taken along line A-A′ of FIG. 60;

FIG. 7 is a schematic diagram illustrating an example of a schematic configuration of an IPS-mode liquid crystal panel according to an embodiment of the present invention.

FIG. 8 is a schematic diagram illustrating an example of a schematic configuration of an FFS-mode liquid crystal panel according to an embodiment of the present invention;

FIG. 9 is a schematic diagram illustrating an example of a schematic configuration of a VA-mode liquid crystal panel according to an embodiment of the present invention; and

FIG. 10 is a schematic diagram of a weight change profile for measuring a boiling point of a solvent for forming an alignment film according to an embodiment of the present invention.

### DETAILED DESCRIPTION OF THE INVENTION

Hereinafter, the present invention will be described in detail together with embodiments (Examples) with reference to the accompanying drawings. Meanwhile, like reference numerals designate like elements throughout the drawings for describing embodiments and the duplicated description is omitted.

First, in a solvent for forming an alignment film in order to form an alignment film including a polymer for a liquid crystal display, a basic structure of a variable compound capable of coating the polymer and changing a chemical structure during a film forming process will be described with reference to the drawings.

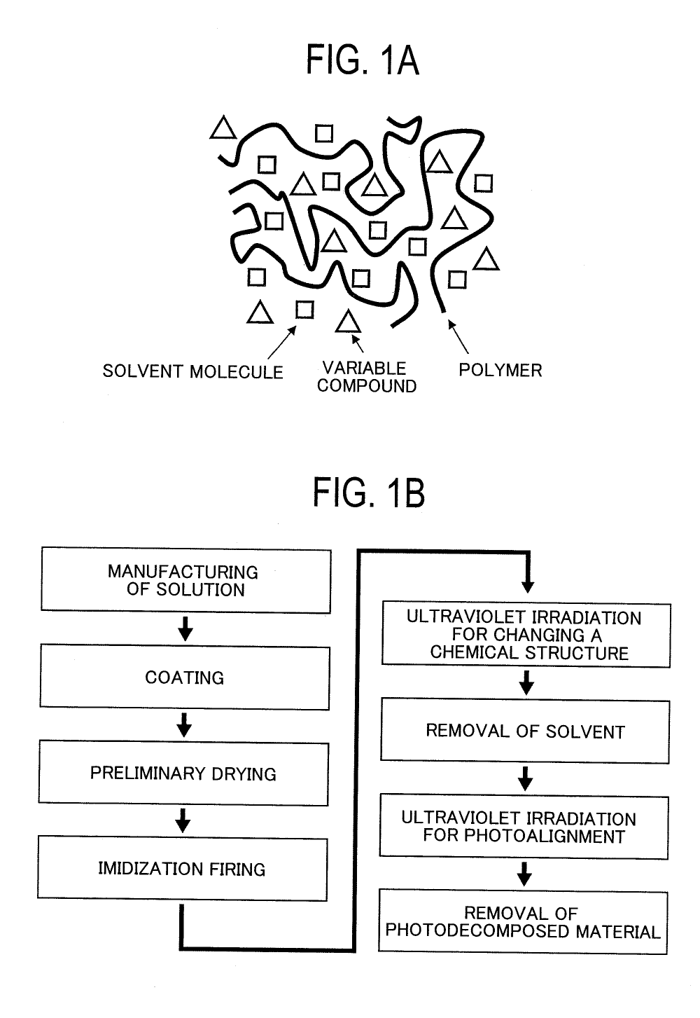

FIG. 1A schematically shows a solution state of a polyamic solution which is a precursor of polyimide as a polymer dissolved in a solvent for forming an alignment film according to an embodiment of the present invention. The solvent for forming an alignment film according to the embodiment includes variable compounds implementing specific functions to be described below and other solvent molecules and polyamic acid is maintained in a solution state by the variable compounds and the solvent molecules.

FIG. 1B shows a block diagram of a process of forming a light alignment film of polyimide by using a solvent for forming an alignment film according to an embodiment of the present invention. First, desired polyamic acid which is a precursor of polyimide is dissolved in a solvent for forming an alignment film according to the embodiment to prepare a solution. Next, the solution is coated on a base substrate forming an alignment film by using a wetting method such as a spin coating, a flexo printing, an inkjet printing, and the like. Subsequently, the coated base substrate is pre-dried so that flatness of a film thickness of the coated solution is not significantly deteriorated by surface tension or unevenness of a base. In this state, while a temperature of a substrate is high, a molecular structure of polyamic acid is thermally changed to be imidized and sintered. Subsequently, ultraviolet light is radiated in order to change a chemical structure of the variable compound included in the solvent for forming an alignment film according to the embodiment. Subsequently, the change is performed and then, the solvent is removed. Thereafter, polarized ultraviolet light for photo-alignment is irradiated on the remaining polyimide thin film and then, a cyclobutane backbone arranged in a polarized direction of main chains of polyimide is photolyzed to cut polyimide chains. Finally, the photolyzed product of polyimide due to the ultraviolet light is removed. Therefore, the photoaligned polyimide alignment film may be fabricated.

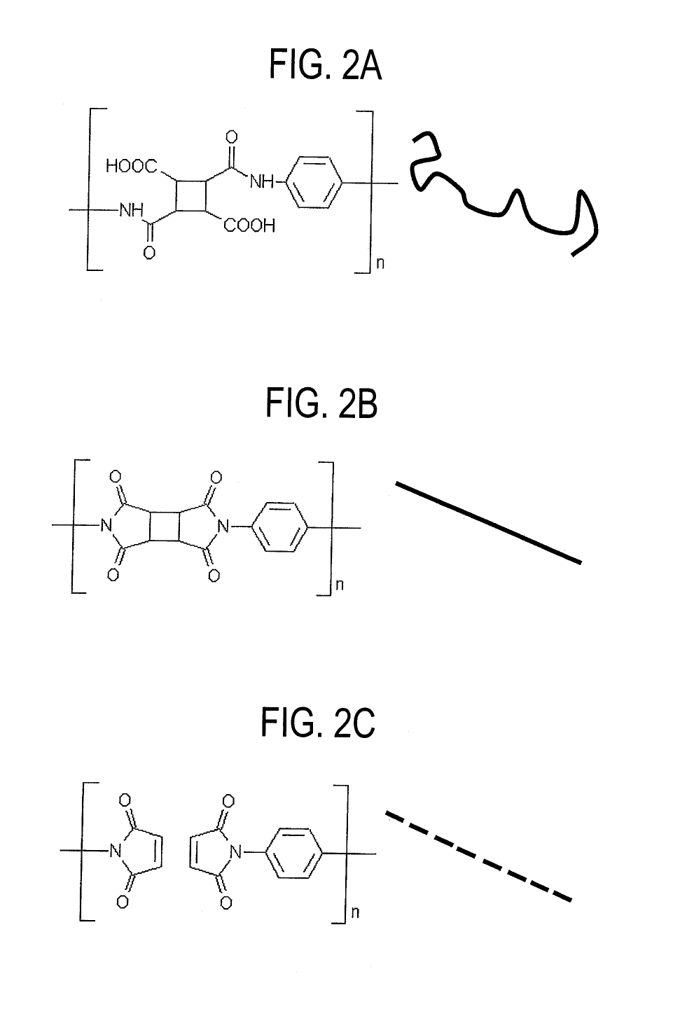

Next, a process of acquiring a high-quality polyimide thin film suitable for photoalignment by using a solvent for forming an alignment film according to the embodiment of the present invention will be described with reference to FIG. 2.

FIG. 2A shows, as an example, a molecular backbone of a polyamic acid polymer synthesized from acid anhydride of a cyclobutane derivative and phenyldiamine. The polyamic acid polymer includes an amide bond —NHCO— on a polymer main chain and in this case, is configured by a repeated structure such as -cyclobutane-CONH-phenylene-NHCO-cyclobutane. Since the amide bond portion can be freely rotated around a carbon-carbon single bond between cyclobutane-CO, a carbon-nitrogen single bond in the amide bond, and a carbon-nitrogen single bond between phenylene-NH, conformation of the main chain of the polymer is changed based on the amide bond and the entire polyamic acid polymer is configured by a polymer having high flexibility and configured by a polymer having a bent band shape.

FIG. 2B shows a molecular backbone of a polyimide polymer when a polyamic acid polymer synthesized from acid anhydride of the cyclobutane derivative and phenyldiamine is completely imidized. When the amide bond is imidized and cyclized, a strong imide backbone (five-membered ring of C—CO—N—CO—C) having high flatness is formed. Accordingly, a bond on the main chain capable of being freely rotated is only a bond between nitrogen of an imide ring and carbon of a phenyl ring and the entire main chain of the polyimide are configured by a strong and high-linearity polymer.

FIG. 2C shows a structure in which the complete polyimide is photolyzed by polarized ultraviolet light. Since polyimide having a phenyl ring on the main chain has a strong dipole moment in the main chain direction, ultraviolet light parallel to the direction is strongly absorbed and ultraviolet light in a vertical direction is not absorbed. The absorption of the phenyl ring is shown as a strong absorption having a peak around a wavelength of 220 to 240 nm and when light of the wavelength is absorbed, the phenyl ring is photo-excited and excited energy thereof moves to the adjacent imide ring and is activated so as to decompose a cyclobutane backbone which is an easily decomposable backbone. Accordingly, the phenyl ring is decomposed from the cyclobutane portion of the polymer main chain. In view of being decomposed by the polarized ultraviolet light, for example, when polyimide is completely aligned in a polarized direction, all the cyclobutane backbones are decomposed, but the complete polyimide in the vertical direction is not decomposed. Accordingly, since polyimide in the vertical direction remains, an alignment film surface configured by polyimide main chains growing in the vertical direction is formed.

As described above, when the complete imidization is progressed, the polyimide main chain of the finally acquired alignment film surface is also configured by a polymer elongated in a specific direction. However, actually, it can be seen that the complete imidization cannot be implemented due to the same reason as follows. That is, as shown in FIG. 2A, the initial polyamic acid is a polymer having high flexibility and the amide bond portion of the polymer is freely bent. Although the alignment film surface is coated with the polyamic acid solution, pre-dried, and has a predetermined film thickness, the polyamic acid is the polymer having high flexibility as it is and a lot of polyamic acid polymers are entangled with each other and has a partially-hardened film form. When the polyamic acid polymers are imidized and sintered by heating in this state, conformation from the imidized portion to the backbone having high linearity is changed, but it is difficult that the strongly-entangled flexible polymer is changed into a completely linear polymer. Accordingly, in order to progress the imidization, a free space and low viscosity are required so that the amide bond is cyclized and linearized in the imide backbone, but the complete imidization is difficult to be implemented. Further, in a known imidization reaction, a solvent having high polarity such as N-methylpyrrolidone (NMP, boiling point of 202° C.), N,N-dimethylformamide, (DMF, boiling point of 153° C.), dimethyl sulfoxide (DMSO, boiling point of 189° C.), and γ-butyrolactone (GBL, boiling point of 204° C.) is used and in order to perform sufficient imidization by heating, since the heating at a temperature of 200° C. or more is required and since the solvent is also evaporated as the imidization progresses, the solvent has gradually high-viscosity, such that the complete imidization is hindered. Accordingly, the completed polyimide has the imide backbone having partially high linearity, but the amide bond portion having high flexibility becomes incomplete polyimide which is not imidized and remains. When the polarized ultraviolet light is irradiated to the incomplete polyimide to perform photolysis, the imide ring portion arranged in the polarized direction may be photolyzed, but the remaining portion as the amide bond having high flexibility is not photolyzed and remains because energy transfer is suppressed from the phenyl ring. Accordingly, one polyimide polymer is in a partially cut state and the remaining polyimide vertical to the polarized direction is also in a partially cut state. Therefore, as compared with the complete polyimide, the alignment film having small surface anisotropy is formed, such that an anchoring characteristic is deteriorated as a liquid crystal alignment film deeply related to the surface anisotropy.

If a remainable solvent having a much higher boiling point is used as the solvent during the imidization, as compared with the case where the known solvent is used, a free space where the main chain of polyamic acid can move even at a high temperature is formed to be further imidized, but in a general solvent having a high boiling point, although the imidization is completed, the solvent remains as the solvent, such that there is no method of removing the solvent. To this end, a process in which a temperature excessively increases, for example, is 400° C. or more or very high vacuum exhaust of 10-3 Torr or less is performed is required. Due to the process, excessive equipment is required for the liquid crystal display to cause a high cost.

However, the solvent for forming the alignment film according to the embodiment is the solvent having a high boiling point up to the imidized sintering, but after the imidized sintering is completed, a chemical structure of the variable compound included in the solvent for forming the alignment film is changed and as a result, the solvent is changed into a solvent having high volatility, such that the solvent can be removed without using the excessively high temperature or the exhaust process. Further, since the chemical structure of the variable compound is changed by light having a wavelength different from the photoalignment, the solvent can be removed without influencing a regulatory force for the alignment. Further, if light of an ultraviolet light source used in the photoalignment is divided and used, two kinds of lights for photoalignment and chemical structure change of the variable compound may be acquired from the same light source and a low cost is implemented without requiring to introduce new light source equipment.

Hereinafter, a basic structure of the variable compound will be described.

A solvent for forming an alignment film in order to form an alignment film including a polymer for a liquid crystal display according to the embodiment is a solvent for forming an alignment film including a variable compound capable of coating the polymer and changing a chemical structure during a film forming process. Further, the solvent for forming the alignment film includes a polymer for a liquid crystal display including polyimide and a variable compound which is in a liquid state during a film forming process of coating and imidized-sintering polyamic acid which is a precursor of polyimide, but changes a chemical structure for evaporating after the imidized-sintering.

In the solvent for forming the alignment film, an example of the variable compound capable of changing the chemical structure is a chemical structure represented by Chemical Formula 1.

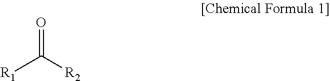

Herein, R₁ is an aromatic carbon compound and R₂ is an aliphatic carbon compound.

An example of R₁ may be a phenyl group as represented by Chemical Formula 2 and a naphthyl group as represented by Chemical Formula 3.

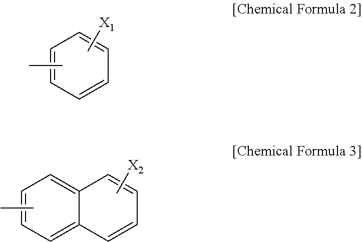

Herein, X₁ or X₂ is a substituent group.

In addition, X₁ or X₂ may be aromatic carbon compounds having a molecular weight or higher, for example, aromatic carbon compounds based on a condensed benzene ring, such as anthracene, tetracene, pentacene, indene, azulene, fluorene, phenanthrene, pyrene, and the like, aromatic carbon compounds based on a bonding structure of a plurality of benzene rings, such as biphenyl, terphenyl, sexiphenyl, and the like, or aromatic carbon compounds based on a complex conjugate system compound, such as furan, pyrrole, pyrazole, pyrazoline, imidazole, oxazole, thiazole, oxadiazole, triazole, thiadiazole, pyran, pyridine, pyridazine, pyrimidine, pyrazine, triazine, benzofuran, benzothiophene, indole, benzimidazole, benzoimidazole, purine, quinoline, isoquinoline, coumarin, shinorine, quinozaline, dibenzofuran, carbazole, acridine, phenanthroline, phenothiazine, flavone, and the like.

Further, an example of R₂ may be a linear alkyl chain as represented by Chemical Formula 4.

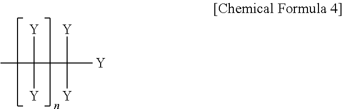

For example, R₂ may be —CH₃, —CH₂CH₃, —CH₂CH₂CH₃, —CH₂CH₂CH₂CH₃, —CH₂CH₂CH₂CH₂CH₃, and the like. In addition, R₂ may be —CH(CH₃)₂, —C(CH₃)₃, —CH₂CH(CH₃)₂, —CH₂C(CH₃)₃, and the like as a branched alkyl chain. In addition, unsaturated bonds —C═C— and —C═C— may be included at a position which does not hinder a change in a chemical structure as described below and hetero atoms such as —O—, -s-, —NH—, and the like may be included in the alkyl chain.

The substituent groups as represented by X₁ of Chemical Formula 2 and X₂ of Chemical Formula 3 may be appropriately introduced to the R₁ and an example of X₁ or X₂ may be a substituent group such as —F, —Cl, —Br, —I, —OH, —SH, —CHO, —COOH, —NH₂, —N(CH₃)₂, —SO₂OH, and the like. In addition, a substituent group configured by an aliphatic carbon compound may also be introduced to the R₁ and on the contrary, a substituent group configured by an aromatic carbon compound may also be introduced to the R₂. The substituent groups may be appropriately introduced at a position which does not hinder a change in a chemical structure as described below.

The R₁ is preferably a phenyl group as represented by Chemical Formula 2 and a naphthyl group as represented by Chemical Formula 3. Further, in the R₂, in the case of Y═—H, n is preferably 1 to 3 and in the case where any one of halogen atoms of —F, —Cl, —Br, and —I is further included in Y, n is preferably 1 to 5.

Further, the solvent for forming an alignment film according to the embodiment is configured by a variable compound in which a boiling point before changing a chemical structure for evaporation is higher than a boiling point after changing the chemical structure. That is, in the process of forming the alignment film as shown in FIG. 1B, since the solvent is in a liquid state up to the imidized-sintering and acts as a solvent for polyamic acid, and may remain although the imidization progresses by heating, a sufficient free volume is given in polyamic acid and a high imidization ratio may be given as compared with a general method. Further, linearity of the polymer main chain may be increased together with the imidization. However, when the solvent remains in the photoalignment, heat fluctuation of the polymer main chain is easy and as a result, the alignment is easily in disorder, such that characteristics such as a surface order parameter, an anchoring force of a liquid crystal, a luminance relaxation constant in a liquid crystal cell state, and the like are deteriorated. In view of the change in the boiling point after and before the change of the chemical structure, it is preferred that a solvent for forming an alignment film is configured by a variable compound having a boiling point before changing the chemical structure for evaporation of 220° C. or more. The reason is because the imidization is sufficiently performed by heating at a temperature of 200° C. or more, and particularly preferably 220° C. or more. The change in the physical properties may vary according to the imidization ratio, the order parameter, the anchoring force, and the luminance relaxation constant and an evaluation method thereof may be a method to be described below.

Further, an example of the change in the chemical structure to be shown by the variable compound of the solvent for forming an alignment film according to the embodiment may use a Norrish decomposition reaction of a carbonyl compound as shown in Chemical Formula 5.

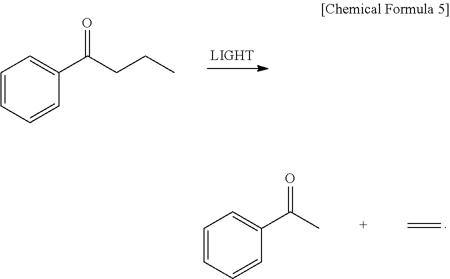

If the compound is used, since buthyrophenone before the chemical structural change has a melting point of 11 to 13° C. and a boiling point of 228 to 230° C., for example, although the imidized-sintering is performed at a temperature of 220° C., buthyrophenone is in a liquid state. However, if light (ultraviolet light having a wavelength of 350 nm or less) is irradiated, buthyrophenon is decomposed into acetophenone and ethylene by Chemical Formula 5. In general, since a boiling point of ethylene is −104° C., ethylene is immediately evaporated and since acetophenone has a melting point of 19 to 20° C. and a boiling point of 202° C., even in the same imidized-sintering temperature of 220° C., the decomposition reaction progresses by further irradiating the ultraviolet light and acetophenone which is a decomposed product may also be evaporated. In a known imidization reaction, a solvent having high polarity such as N-methylpyrrolidone (NMP, boiling point of 202° C.), N,N-dimethylformamide, (DMF, boiling point of 153° C.), dimethyl sulfoxide (DMSO, boiling point of 189° C.), and γ-Butyrolactone (GBL, boiling point of 204° C.) is used. In order to maintain film-formation of polyamic acid, an appropriately mixed solvent in the known form is preferably used and even at a high temperature in which the solvents are evaporated, since the solvents still remain as a liquid component, a desired function may be achieved. That is, the solvent for forming an alignment film according to the embodiment includes a variable compound of which a molecular weight before a chemical structural change for evaporation is larger than a molecular weight after the chemical structural change. In addition, the solvent includes a variable compound having different polarities before and after the chemical structural change for evaporation. Decarbonylation of a carbonyl compound, denitrogenation of an azo compound, a silane coupling reaction having a nitrobenzyl group, a two-photon absorption/decomposition reaction of a bisphenyldisulfide compound, and the like may also be used in the chemical structural change. In the case where a material using the photoreaction is used, an optimal basic structure of a variable compound is also selected from the structure other than Chemical Formula 1. In either case, the solvent for forming an alignment film according to the embodiment includes the variable compound which changes the chemical structure by irradiating light from the outside and preferably, the variable compound which changes the chemical structure by irradiating light having a wavelength of 400 nm or less from the outside.

In the solvent for forming an alignment film according to the embodiment, the variable compound is preferably evaporated by heating in order to remove the compound after changing the chemical structure. Particularly, as described above, it is preferred that the compound is removed by heating at a temperature lower than a temperature for imidization due to the heating and more particularly, it is preferred that the compound is evaporated by heating at room temperature or more to a temperature of 230° C. or less. Further, in order to more efficiently remove the compound after changing the chemical structure, the compound may be evaporated by depressurizing and particularly, is preferably evaporated by pressurizing at a pressure of 1 mmTorr or less. In addition, in order to efficiently remove the compound after changing the chemical structure, an air current having an appropriate temperature may flow on a film surface. In addition, the compound after changing the chemical structure may be removed with a different solvent, but it is disadvantageous due to a cost in that a separate solvent is required.

Further, the solvent for forming an alignment film is a solvent for forming polyimide forming an alignment film, in which an alignment regulatory force of the liquid crystal is given after evaporating the solvent for forming an alignment film. Particularly, as a method of giving the alignment regulatory force, it is preferred that an alignment film, in which the alignment regulatory force of the liquid crystal is given by the polarized light, is formed. Furthermore, it is preferred that the solvent for forming an alignment film is a solvent for forming polyimide forming an alignment film, in which the alignment regulatory force of the liquid crystal is given by the polarized light having a different wavelength from the light changing the chemical structure of the variable compound. In addition, it is preferred that the solvent for forming an alignment film is a solvent for forming polyimide forming an alignment film, in which the alignment regulatory force of the liquid crystal is given by the polarized light having a wavelength smaller than a wavelength of the light changing the chemical structure of the variable compound. By selectively selecting the irradiating light, at the same substrate heating temperature, a film-forming process may be controlled only by replacing light.

Further, it is preferred that the solvent for forming an alignment film according to the embodiment is a solvent for forming polyimide forming an alignment film, in which the alignment regulatory force of the liquid crystal is given by cutting a part of the main chain by the polarized light. By selecting the material, proven polyimide may be used as the liquid crystal alignment film.

Further, in the solvent for forming an alignment film according to the embodiment, in view of high performance of the final alignment film, it is preferred that a part of the structure changing the chemical structure of the variable compound is a chemical structure capable of remaining in the alignment film when the solvent for forming an alignment film is evaporated. For example, as the chemical structure capable of remaining in the alignment film, when a chemical structure, which is transparent with respect to visible light, but has absorption with respect to ultraviolet light, is used, a ultraviolet filter function may be given to the alignment film itself.

Further, as described above, the solvent for forming an alignment film according to the embodiment is preferably a solvent for forming polyimide which forms an alignment film having an alignment regulatory force, in which the liquid crystal is aligned in the film plane by an alignment regulatory force after evaporating the solvent for forming an alignment film. In addition, the solvent for forming an alignment film is preferably a solvent for forming polyimide which forms an alignment film having a regulatory force, in which the liquid crystal is aligned in the film plane in a standing direction of 30 degrees or more by an alignment regulatory force. The direction of the alignment regulatory force is set so as to be a desirable direction in various display modes of the liquid crystal display to be described below, but in order to further increase the alignment direction or to generate the pretilt angle, a part of the structure having the changed chemical structure of the variable compound may intentionally remain in the alignment film. Further, by manufacturing the liquid crystal display by using the solvent for forming an alignment film according to the embodiment, it is possible to provide a liquid crystal display having high anchoring, high contrast, and low afterimage as compared with the known method.

A nature of the alignment film having the high-quality by the solvent for forming an alignment film according to the embodiment may be characterized by an imidization ratio, an order parameter, an anchoring force, and a luminance relaxation constant thereof.

First, the imidization ratio of the alignment film was measured by using 1H-NMR around the amide bond as can be clearly seen by comparison between FIGS. 2A and 2B. That is, a peak integral value derived from proton of carboxylic acid around polyamic acid or a peak integral value derived from proton of the amide bond portion was calculated by using a solid NMR and when in the case of the initial film value without imidization, the imidization ratio was 0% and in the case of the peak strength of 0, the imidization ratio was 100%, a cyclization ratio thereof was calculated.

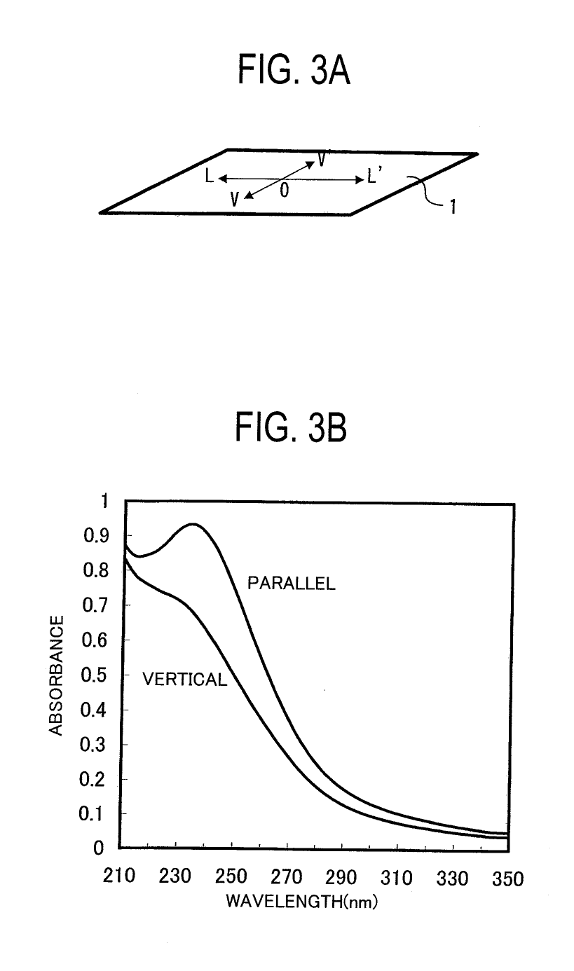

Further, the order parameter of the alignment film was measured by the following method. One method is a method of measuring absorption anisotropy in the film plane of the alignment film. FIG. 3A shows a model diagram of an alignment film in which anisotropy is generated in the film plane by photoalignment processing. When the polyimide chain is cut by the polarized ultraviolet light, since a transition moment is included in the main chain direction of a phenyl ring of the polymer, the light in the direction is strongly absorbed, a cyclobutane ring of the polymer main chain in the direction is opened, and the main chain is cut (see FIG. 2C). In contrast, since the main chain in a direction perpendicular to the polarized ultraviolet light is not cut, the main chain remains in the polymer state. As a result, in the photoalignment-processed alignment film, the polymer main chain in the direction perpendicular to the irradiation direction of the polarized ultraviolet light remains (the direction is referred to as line L-L′) and the polymer main chain in the parallel direction is cut (the direction is referred to as line V-V′) and a relationship therebetween is 90 degrees based on a center point O of the film. FIG. 3B shows an example measuring a spectrophotometry using a polarized light source with respect to the photoalignment-processed alignment film. FIG. 3B shows wavelength dependence of absorption when the polarized direction of the polarized light source is parallel to the direction L-L′ in which the polymer main chain remains (disclosed as “parallel” in FIG. 3B) and when the polarized direction of the polarized light source is vertical to the direction L-L′ in which the polymer main chain remains (disclosed as “vertical” in FIG. 3B) by rotating the thin film by 90 degrees based on the center point O. The absorption peak having the wavelength of 233 nm is shown and the strength of the “parallel” is stronger than the strength of “vertical”. When the absorption in the peak wavelength is represented by Iₚₐᵣₐₗₗₑₗ and I_{vertical} the order parameter S_{film} due to the light absorption of the film is defined by the following Equation 1.

$${S}_{FILM} = \frac{{I}_{PARALLEL} - {I}_{VERTICAL}}{{I}_{PARALLEL} + 2 {I}_{VERTICAL}} \qquad [ Equation 1 ]$$

As described above, when the absorption spectrum can be measured in only the film, the order parameter may be calculated from the anisotropy of the absorption.

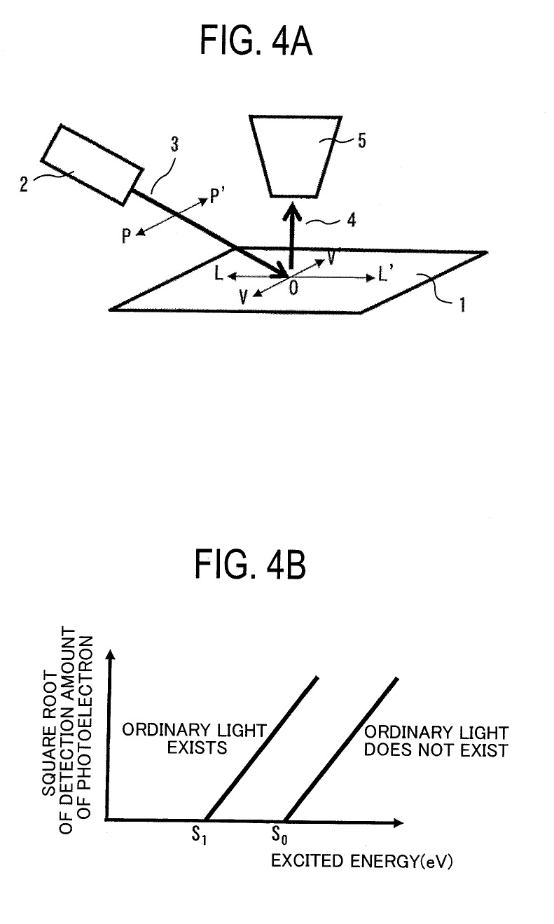

Another method of measuring the order parameter uses anisotropy of emission of photoelectrons of the film. The above-mentioned method is a method dependent to the absorption of the film and particularly, in order to absorb the light of the ultraviolet area, the substrate including the alignment film should be made of an ultraviolet-transmittable material, for example, synthetic quartz. In addition, in the actual liquid crystal panel, various members such as a TFT circuit, an intermediate insulating film, a wiring, and the like are compactly mounted between the alignment film and the substrate. As a result, it is difficult to measure the absorption spectrum in only the alignment film. Therefore, by the anisotropic absorption of a phenyl ring in the polymer main chain direction, an excited state is generated in the direction and further transited into a high energy state by irradiation the second light therein to discharge the photoelectrons, such that the order parameter of the alignment film surface was evaluated. As shown in FIG. 4A, when light polarized in one direction by using a polarizer is irradiated toward the excited light source 2 and the surface of the alignment film 1 by using an low-energy electronic spectrometer (manufactured AC-2 by RIKEN KEIKI Co., Ltd.), using a spectrum light source (spectrometry of heavy hydrogen lamp by a diffraction grid, wavelength of 364 to 200 nm, quantity of light of 50 nW, and scanning excited energy from a low side to a high side) and a normal light source (He—Ne laser, wavelength of 632.8 nm, and quantity of light of 2 mW) as a light source, and collimating both the light sources as the same light path, the polarized light was referred to as an excited light 3. Herein, the polarized direction of the excited light 3 is represented by line P-P′ and line P-P′ is parallel to the film plane. In addition, (although not particularly shown) the optical layout of the spectrum light source and the normal light source was optimized so that the strengths of the spectrum light source and the normal light source were maximized in the polarized direction. As a result, when the excited light 3 is irradiated in the alignment film 1, photoelectrons 4 start to be discharged from the light of the wavelength of the spectrum light source shorter than any wavelength and the number of the photoelectrons per unit time is counted by a photoelectron detector 5 disposed directly on the alignment film, such that the detected number of the photoelectrons was referred to as a photoelectron detecting amount. FIG. 4B schematically shows the square root of the acquired photoelectron detecting amount and excited energy of the spectrum light source (=1240/wavelength of spectrum light source [nm], unit: eV). For example, as shown in FIG. 3B, in the alignment film configured by polyimide, in the case where the normal light source is not used, when the wavelength of the spectrum light source is shorter than the wavelength of 215 nm, the photoelectrons start to be detected (a position of S0) and in the case where the normal light source is used, the photoelectrons start to be detected from the vicinity of the wavelength of 310 nm which is longer than the wavelength thereof, (a position of S1). The light is generally absorbed by only the excited light source, but the energy discharging the photoelectrons from the film is deficient. Accordingly, it is considered that the excited electron by the light absorption is further excited up to high energy by adding the normal light source to discharge the photoelectrons. In order to evaluate the order parameter, when the wavelength of the excited light source is fixed as a wavelength having maximal anisotropy of the absorption spectrum, for example, the peak wavelength of 232 nm in the case of the material shown in FIG. 3B and the normal light is used, the square root Jₚₐᵣₐₗₗₑₗ of the photoelectron detecting amount when the polarized direction P-P′ of the excited light is parallel to the light alignment direction L-L′ of the alignment film and the square root J_{vertical} of the photoelectron detecting amount when the polarized direction P-P′ of the excited light is parallel to the direction V-V′ vertical to the light alignment direction of the alignment film are measured, and the order parameter S_{surface} of the alignment film is determined by the following Equation 2.

$${S}_{FILM} = \frac{{J}_{PARALLEL} - {J}_{VERTICAL}}{{J}_{PARALLEL} + 2 {J}_{VERTICAL}} \qquad [ Equation 2 ]$$

Since the ejecting depth of the photoelectron emission is about several nm, the anisotropy of the polymer main chain of only a film surface may be detected and there is no influence of the base. Further, although the liquid crystal is sealed as a product, the order parameter is measured after the liquid crystal panel is released and the liquid crystal attached to the alignment film surface is washed with a solvent which does not intrude into the alignment film such as hexane, such that the order parameter may be evaluated.

Subsequently, the anchoring force was measured by the following method. A homogenous alignment liquid crystal cell for evaluation is fabricated by coating the alignment film on two sheets of glass substrates as a set to perform the photoalignment processing, and interposing a spacer having an appropriate thickness d so that the alignment directions of the two sheets of alignment films are parallel to each other. A nematic liquid crystal material (spiral pitch p and elastic constant K₂) containing a chiral agent of which a material property is known is sealed therein and the cell for evaluation is maintained once in a liquid crystal isotropic phase in order to stabilize the alignment, and then, at room temperature, a twist angle φ₂ is measured by the following method. Subsequently, after most of the liquid crystal in the cell is removed by an air pressure or a centrifugal force and the inner cell is solvent-washed and dried, a portion without the chiral agent is sealed with the same liquid crystal, and the alignment is stabilized, and then, the twist angle φ₁ is measured. In this case, the anchoring strength is defined by the following Equation 3.

$${A}_{φ} = \frac{2 {K}_{2} ( 2 π d / p - {φ}_{2} )}{d sin ( {φ}_{2} - {φ}_{1} )} \qquad [ Equation 3 ]$$

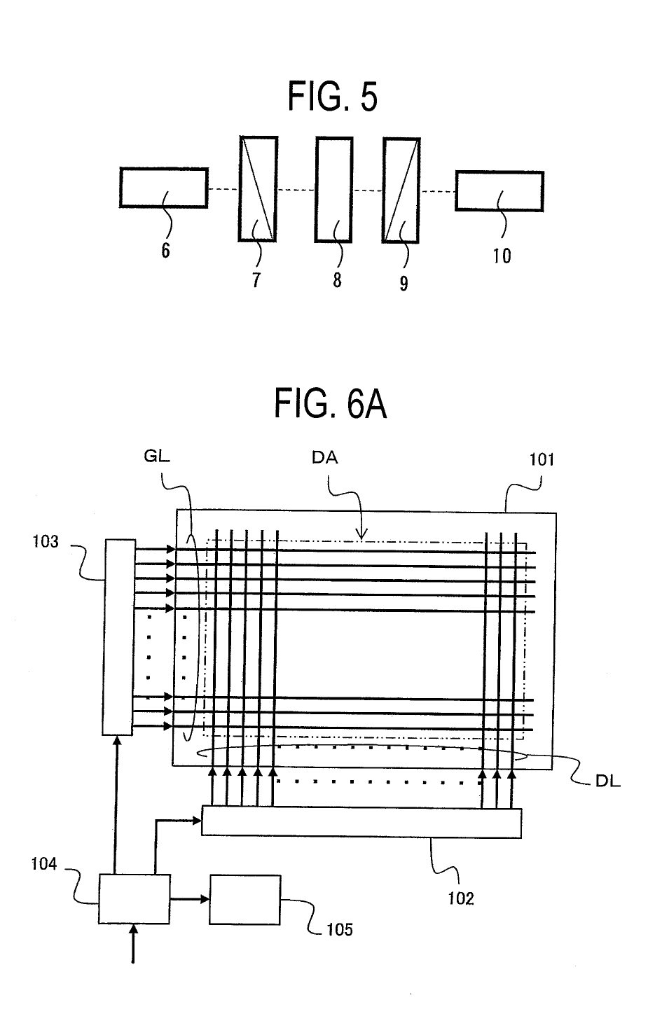

Further, the twist angle was measured by using an optical system as shown in FIG. 5. That is, a visible light source 6 and a photomultiplier tube 10 are collimated on the same straight line and a polarizer 7, a cell for evaluation 8, and an analyzer 9 are disposed therebetween in sequence. A tungsten lamp is used as the visible light source 6 and a transmitting axis of the polarizer 7 and an absorbing axis of the analyzer 9 are combined so as to be substantially parallel to the alignment direction L-L′ of the alignment film of the cell for evaluation 8. Subsequently, only the polarizer is rotated and the angle is changed so that strength of the transmitting light is minimized. Subsequently, only the analyzer is rotated and the angle is changed so that strength of the transmitting light is minimized. Hereinafter, similarly, the rotation of only the polarizer and the rotation of only the analyzer are repeated until the angle is constant. Finally, at the convergence, a rotation angle φ_{polarizer} of the transmitting axis of the polarizer and a rotation angle φ_{analyzer} of the absorbing axis of the analyzer are defined as a twist angle φ=φ_{analyzer}−φ_{polaraizer}. Herein, a measurement error may be reduced by controlling a refraction index anisotropy Δn of the used liquid crystal and a thickness d of the liquid crystal cell.

Subsequently, the luminance relaxation constant was determined by the following method. According to an order as described below in detail, various liquid crystal display elements including an alignment film are fabricated. Black and white window patterns are continuously displayed for a predetermined time (called a printing time) on the liquid crystal display and then, immediately converted into display voltage of a gray level which is an intermediate gray of the entire screen, such that a time at which the window patterns (called afterimages) are lost is measured. Ideally, in the alignment film, since a remaining charge is not generated in any portion of the liquid crystal display and the direction of the alignment regulatory force is not disordered, the gray level of the entire screen is immediately displayed together with the conversion of the display voltage. However, since an effective alignment state of a bright area (a white pattern region) is deviated from an ideal level due to the generation of the remaining charge and the disorder of the direction of the alignment regulatory force according to driving, the luminance looks different, but if the voltage of the intermediate gray display is maintained for a long time, the remaining charge in the voltage or the alignment regulatory force direction is stabilized to be uniformly displayed. A time until a luminance distribution in a plane of the liquid crystal element is measured by a CCD camera and the uniform display is implemented is referred to as an afterimage time and the luminance relaxation constant of the liquid crystal display element is defined by the afterimage time.

Hereinafter, a liquid crystal display including a high-quality alignment film fabricated by the solvent for forming an alignment film according to an embodiment of the present invention will be described.

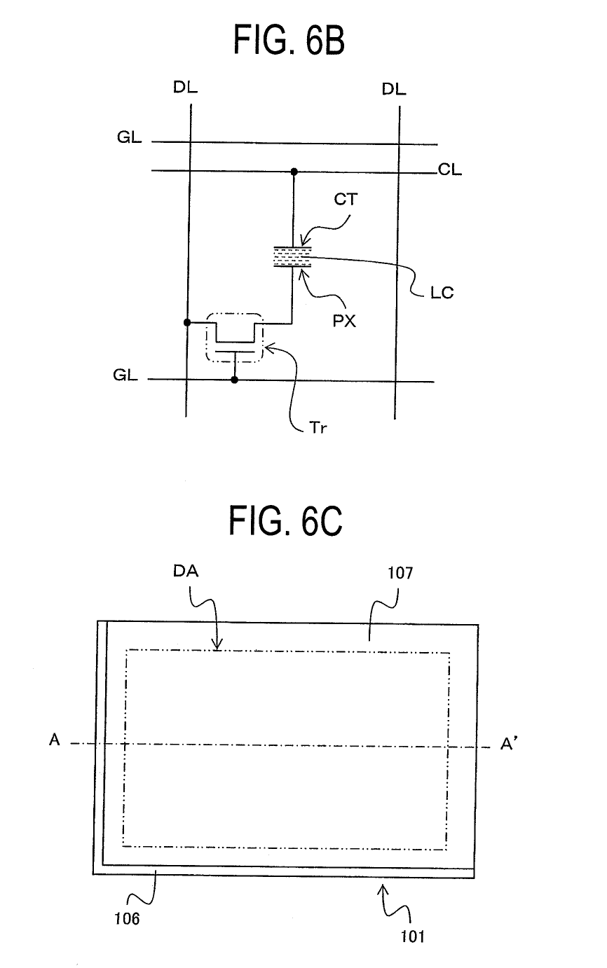

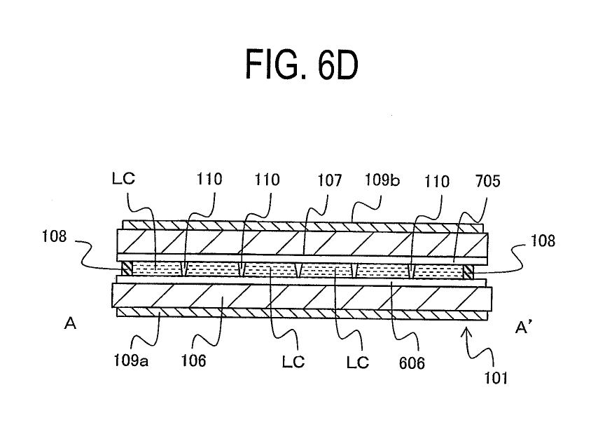

FIGS. 6A to 6D are schematic diagrams illustrating an example of a schematic configuration of a liquid crystal display according to an embodiment of the present invention. FIG. 6A is a schematic block diagram illustrating an example of a schematic configuration of a liquid crystal display according to an embodiment of the present invention. FIG. 6B is a schematic circuit diagram illustrating an example of a circuit configuration of one pixel of a liquid crystal panel. FIG. 6C is a schematic plan view illustrating an example of a schematic configuration of a liquid crystal panel. FIG. 6D is a schematic cross-sectional view illustrating an example of a cross-sectional configuration taken along line A-A′ of FIG. 6C.

A high-quality alignment film fabricated by the solvent for forming an alignment film according to an embodiment of the present invention is applied to, for example, an active matrix mode liquid crystal display. The active matrix mode liquid crystal display is used for, for example, a display (monitor) for portable electronic appliances, a display for PC, a display adapted for printing or design, a display for medical equipment, a liquid crystal television, and the like.

As shown in FIG. 6A, the active matrix mode liquid crystal display includes, for example, a liquid crystal panel 101, a first driving circuit 102, a second driving circuit 103, a control circuit 104, and a backlight 105.

The liquid crystal panel 101 includes a plurality of scanning signal lines (gate lines) GL and a plurality of image signal lines (drain lines) DL, and the image signal lines DL are connected to the first driving circuit 102 and the scanning signal lines GL are connected to the second driving circuit 103. In addition, FIG. 6A shows some of the plurality of scanning signal lines GL are shown and a plurality of scanning signal lines GL are more densely disposed in the actual liquid crystal panel 101. Similarly, FIG. 6A shows some of the plurality of image signal lines DL and a plurality of image signal lines DL are more densely disposed in the actual liquid crystal panel 101.

Further, a display area DA of the liquid crystal panel 101 is configured by a set of a plurality of pixels and a region occupied by one pixel in the display area DA corresponds to, for example, a region surrounded by two adjacent scanning signal liens GL and two adjacent image signal lines DL. In this case, a circuit of one pixel may be configured, for example, as shown in FIG. 6B and the circuit includes a TFT element Tr acting as an active element, a pixel electrode PX, a common electrode CT (also, referred to as an opposed electrode), and a liquid crystal layer LC. Further, a common line CL for commonalizing common electrodes CT of the plurality of pixels is disposed in the liquid crystal panel 1.

Further, the liquid crystal panel 101 includes alignment films 606 and 705 formed on the surfaces of an active matrix substrate 106 and an opposed substrate 107 and a liquid crystal layer LC (liquid crystal materials) interposed between the alignment films, as shown in FIGS. 6C and 6D. Further, although not particularly shown therein, an intermediate layer (for example, an optical intermediate layer such as a retardation film, a color conversion layer, a light diffusion layer, or the like) may be appropriately disposed between the alignment film 606 and the active matrix substrate 106 or between the alignment film 705 and the opposed substrate 107. In this case, the active matrix substrate 106 and the opposed substrate 107 are bonded with each other by a ring-shaped seal member 108 disposed at the outside of the display area DA and the liquid crystal layer LC is sealed by a space surrounded by the alignment film 606 of the active matrix substrate 106, the alignment film 705 of the opposed substrate 107, and the seal member 108. Further, the liquid crystal panel 101 of the liquid crystal display including the backlight 105 includes an active matrix substrate 106, a liquid crystal layer LC, and a pair of polarizers 109a and 109b facing each other with an opposed substrate 107 interposed therebetween.

Meanwhile, the active matrix substrate 106 is a substrate in which scanning signal lines GL, image signal lines DL, active elements (TFT elements Tr), pixel electrodes PX, and the like are disposed on an insulation substrate such as a glass substrate and the like. Further, when a driving mode of the liquid crystal panel 101 is an in-plane switching mode such as an IPS mode, the common electrode CT and the common line CL are disposed on the active matrix substrate 106. Further, when a driving mode of the liquid crystal panel 101 is a vertical switching mode such as a TN mode or a vertical alignment (VA) mode, the common electrode CT is disposed on the opposed substrate 107. In the case of the vertical switching mode liquid crystal panel 101, generally, the common electrode CT is a sheet of large-area flat electrode which is shared in all the pixels and the common line CL is not disposed.

Further, in the liquid crystal display according to the embodiment, a plurality of columnar spacers 110 for uniformizing a thickness (also, referred to as a cell gap) of the liquid crystal layer LC in each pixel are disposed at a space where the liquid crystal layer LC is sealed. The plurality of columnar spacers 110 may be disposed on the opposed substrate 107.

The first driving circuit 102 is a driving circuit which generates image signals (also, referred to as gray voltage) applied to the pixel electrode PX of each pixel through the image signal line DL and generally, is called a source driver, a data driver, or the like. Further, the second driving circuit 103 is a driving circuit which generates scanning signals applied to the scanning signal lines GL and generally, is called a gate driver, a scanning driver, or the like. Further, the control circuit 104 performs a control of an operation of the first driving circuit 102, a control of an operation of the second driving circuit 103, a control of luminance of the backlight 105, and the like and generally, is a control circuit called a TFT controller, a timing controller, or the like. Further, the backlight 105 may be a light source such as, for example, a fluorescent lamp such as a cold cathode fluorescent lamp, a light emitting diode (LED), or the like and the light emitted by the backlight 105 is converted into planar light by a reflective plate, a light guide plate, a light diffusion plate, a prism sheet, or the like (not shown) to be irradiated to the liquid crystal panel 101.

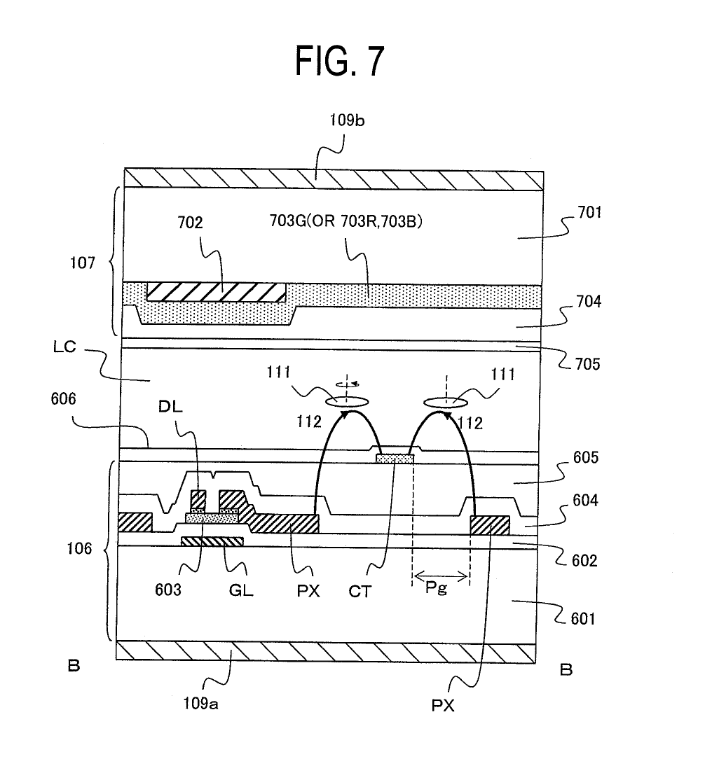

FIG. 7 is a schematic diagram illustrating an example of a schematic configuration of an IPS mode liquid crystal panel according to an embodiment of the present invention. The active matrix substrate 106 includes scanning signal lines GL, a common line CL, and a first insulating layer 602 covering the scanning signal lines and the common line which are formed on the surface of the insulation substrate such as a glass substrate 601. A semiconductor layer 603 of the TFT element Tr, an image signal line DL, a pixel electrode PX, and a second insulating layer 604 covering the semiconductor layer, the image signal line, and the pixel electrode are formed on the first insulation layer 602. The semiconductor layer 603 is disposed on the scanning signal line GL and a portion of the scanning signal line GL disposed below the semiconductor layer 603 functions as a gate electrode of the TFT element Tr. In addition, the semiconductor layer 603 is configured by stacking a source diffusion layer and a drain diffusion layer which are made of the second amorphous silicon having different kind and concentration of impurity from the first amorphous silicon, on an active layer (channel forming layer) made of the first amorphous silicon. Further, a part of the image signal line DL and a part of the pixel electrode PX are disposed on the semiconductor layer 603 and the parts disposed on the semiconductor layer 603 act as the drain electrode and the source electrode of the TFT element Tr. However, the source and the drain of the TFT element Tr are changed according to a bias relationship, that is, a relationship in height between a potential of the pixel electrode PX and a potential of the image signal line DL when the TFT element Tr is turned on. However, hereinafter, an electrode connected to the image signal line DL is referred to as the drain electrode and an electrode connected to the pixel electrode is referred to as the source electrode. A third insulating layer (an overcoat layer) 605 having a flat surface is formed on the second insulation layer 604. The common electrode CT and an alignment film 606 covering the common electrode CT and the third insulation layer 605 are formed on the third insulation layer 605. The common electrode CT is connected with the common line CL via a contact hole CH (a through-hole) passing through the first insulation layer 602, the second insulation layer 604, and the third insulation layer 605. In addition, the common electrode CT may be formed so as to have a gap Pg of about 7 μm between the common electrode CT and the pixel electrode PX in a plane. The alignment film 606 is coated with polymer materials described in the Examples below and a surface processing (a rubbing processing or the like) for giving a liquid crystal alignment ability is performed on the surface thereof. Meanwhile, the opposed substrate 107 includes a black matrix 702, color filters 703R, 703G, and 703B, an overcoat layer 704 covering the black matrix and the color filters which are formed on the surface of an insulation substrate such as a glass substrate 701 or the like. The black matrix 702 may be a light blocking film having a grid shape for providing an opening region of a pixel unit in the display area DA. Further, the color filters 703R, 703G, and 703B may be a film which transmits only light having a specific wavelength region (color) of white light from the backlight 105 and when the liquid crystal display corresponds to an RGB mode color display, a color filter 703R transmitting red light, a color filter 703G transmitting green light, and a color filter 703B transmitting blue light are disposed (herein, one color pixel is representatively shown). Further, the overcoat layer 704 has a flat surface. A plurality of columnar spacers 110 and the alignment film 705 are formed on the overcoat layer 704. The columnar spacer 110 is, for example, a conical trapezoid having a flat ridge (also, referred to as a trapezoidal rotating body) and is disposed at a position overlapping with a portion except for a portion where the TFT element Tr is disposed and a portion crossing the image signal line DL at the scanning signal line CL of the active matrix substrate 106. Further, the alignment film 705 is made of, for example, a polyimide-based resin and a surface processing (a rubbing processing or the like) for giving a liquid crystal alignment ability is performed on the surface thereof.

Further, in the liquid crystal panel 101 of FIG. 7, liquid crystal molecules 111 of the liquid crystal layer LC are aligned to be substantially parallel to the surfaces of the glass substrates 601 and 701 when the electric field having the same potential as the pixel electrode PX and the common electrode CT is not applied and the liquid crystal molecules 111 are homogenously aligned in an initial alignment direction defined by the rubbing processing performed on the alignment films 606 and 706. In addition, while the TFT element Tr is turned on, when gray voltage applied to the image signal line DL is inputted to the pixel electrode PX and a potential difference between the pixel electrode PX and the common electrode CT is generated, an electric field 112 (electric force line) is generated as shown in FIG. 7 and the electric field 112 having strength according to the potential difference between the pixel electrode PX and the common electrode CT is applied to the liquid crystal molecules 111. In this case, since the direction of the liquid crystal molecules 111 configuring the liquid crystal layer LC is changed into a direction of the electric field 112 by an interaction between dielectric anisotropy of the liquid crystal layer LC and the electric field 112, refraction anisotropy of the liquid crystal layer LC is changed. In this case, the direction of the liquid crystal molecules 111 is determined by strength of the applied electric field 112 (a magnitude of the potential difference between the pixel electrode PX and the common electrode CT). Accordingly, the liquid crystal display may display images by controlling the gray voltage applied to the pixel electrode PX for each pixel with a fixed potential of the common electrode CT to change transmittance of light in each pixel.

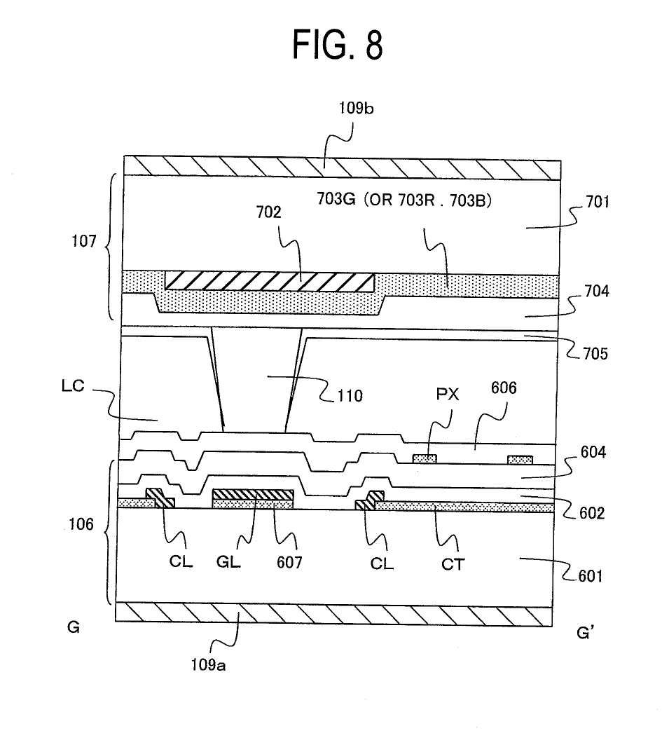

FIG. 8 is a schematic diagram illustrating an example of a schematic configuration of an FFS mode liquid crystal panel according to an embodiment of the present invention. The active matrix substrate 106 includes common electrodes CT, scanning signal lines GL, common lines CL, and a first insulating layer 602 covering the common electrodes CT, the scanning signal lines GL, and the common lines CL which are formed on the surface of the insulation substrate such as a glass substrate 601. A semiconductor layer 603 of the TFT element Tr, an image signal line DL, a source electrode 607, and a second insulating layer 604 covering the semiconductor layer, the image signal line, and the source electrode are formed on the first insulation layer 602. In this case, a part of the image signal line DL and a part of the source electrode 607 are disposed on the semiconductor layer 603 and the parts disposed on the semiconductor layer 603 (herein, not shown because the parts are hidden in a depth direction) act as the drain electrode and the source electrode of the TFT element Tr. Further, the third insulating layer 605 is not formed in the liquid crystal panel 101 of FIG. 8 and the pixel electrode PX and the alignment film 606 covering the pixel electrode PX is formed on the second insulating layer 604. The pixel electrode PX is connected with the source electrode 607 via a contact hole CH (a through-hole) passing through the second insulating layer 604. In this case, the common electrode CT formed on the surface of the glass substrate 601 is formed in a flat shape in a region (opening region) surrounded by two adjacent scanning signal lines CL and two adjacent image signal lines DL and the pixel electrodes PX having a plurality of slits are stacked on the flat common electrode CT. Further, the common electrodes CT of the pixels arranged in an extending direction of the scanning signal line GL are commonalized by the common line CL. Meanwhile, the opposed substrate 107 in the liquid crystal panel 101 of FIG. 8 has the same configuration as the opposed substrate 107 in the liquid crystal panel 101 of FIG. 7. Therefore, the detailed description for the configuration of the opposed substrate 107 is omitted.

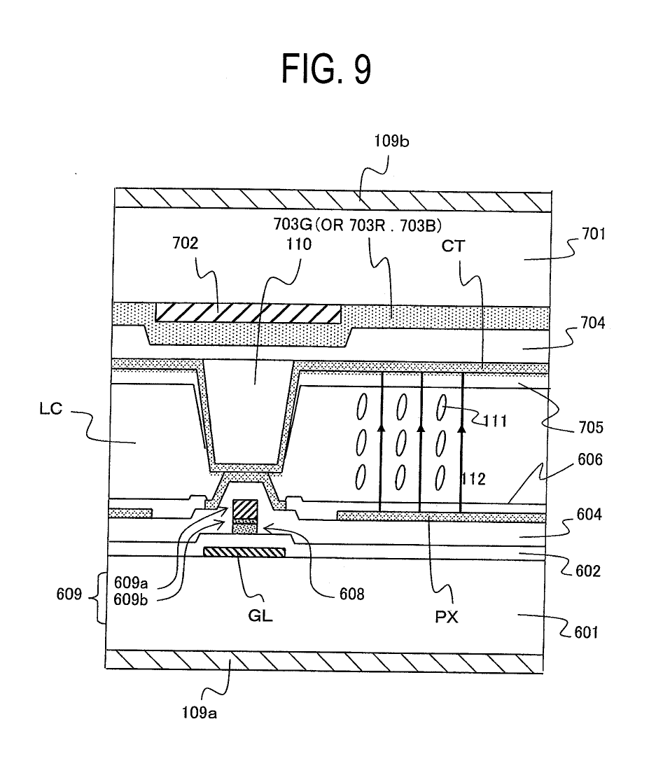

FIG. 9 is a schematic cross-sectional view illustrating an example of a cross-sectional configuration of a main part of a VA-mode liquid crystal panel according to an embodiment of the present invention. In the vertical switching mode liquid crystal panel 101, as shown in FIG. 9, the pixel electrode PX is formed on the active matrix substrate 106 and the common electrode CT is formed on the opposed substrate 107. In the case of the VA mode liquid crystal panel 101 which is one of the vertical switching modes, the pixel electrode PX and the common electrode CT is formed in a beta shape (a simple flat shape) by a transparent conductor of ITO or the like. In this case, the liquid crystal molecules 110 are arranged to be perpendicular to the surfaces of the glass substrates 601 and 701 by the alignment films 606 and 705 when the same electric field as the potential difference between the pixel electrode PX and the common electrode CT is not applied. In addition, when the potential difference between the pixel electrode PX and the common electrode CT is generated, the electric field 112 (the electric force line) substantially perpendicular to the glass substrates 601 and 701 is generated and the liquid crystal molecules 111 are fallen in a direction parallel to the substrates 601 and 701, such that a polarized state of incident light is changed. In this case, the direction of the liquid crystal molecules 111 is determined by strength of the applied electric field 112. Accordingly, the liquid crystal display displays images by controlling the image signal (gray voltage) applied to the pixel electrode PX for each pixel with a fixed potential of the common electrode CT to change transmittance of light in each pixel. Further, in the configuration of the pixel in the VA mode liquid crystal panel 101, various configurations of the flat shape of the TFT element Tr or the pixel electrode PX are known and the configuration of the pixel of the liquid crystal panel 101 of FIG. 9 may be one of the configurations. Herein, the detailed description for the configuration of the pixel in the liquid crystal panel 101 is omitted.

The embodiment of the present invention relates to a portion contacted to the liquid crystal layer LC and a peripheral configuration thereof in the liquid crystal panel 101, particularly, the active matrix substrate 106 and the opposed substrate 107 of the active matrix mode liquid crystal display. Therefore, the detailed description for the configuration of the first driving circuit 102, the second driving circuit 103, the control circuit 104, and the backlight 105 that do not directly relate to the present invention is omitted.

In order to manufacture the liquid crystal displays, various alignment materials or alignment processing methods, various liquid crystal materials, and the like which are used in the liquid crystal display may be used and may be applied to various processes when the liquid crystal display is assembled and processed.

Hereinafter, the present invention will be described in detail using Examples, but the technical scope of the present invention is not limited to the Examples to be described below.

### Example 1

First, as the solvent for forming an alignment film in order to form the alignment film including a polymer for a liquid crystal display according to the embodiment of the present invention, the solvent for forming an alignment film including butyrophenone, which is a material having a chemical structure of Chemical Formula 6 in the variable compound capable of changing the chemical structure during a film-forming process after coating the polymer, is used, such that an example in which an imidization ratio is improved will be described.

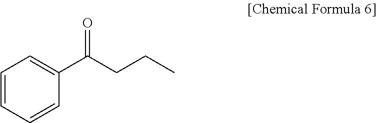

In the used polymer for an alignment film, polyamic acid which may finally become polyimide as shown in Chemical Formula 7 by the imidization was used as a raw material.

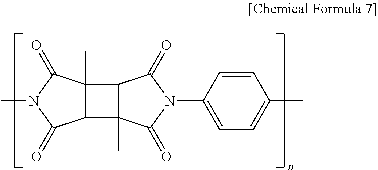

First, γ-butyrolactone (GBL, boiling point of 204° C.) of 10 wt % was further added in a solution dissolving polyamic acid of 20 wt % in N-methylpyrrolidone (NMP, boiling point of 202° C.) to be used as an undiluted solution of polyamic acid. A uniform thin film may be formed by a spin coating method or a printing method when using the undiluted solution. A mixed solution was fabricated by adding the variable compound of Chemical Formula 6 at a rate of 0 to 90 wt % in the undiluted solution (the compound has a melting point of 11° C. and a boiling point of 230° C., has no risk of being precipitated as a solid during the film-forming process up to the imidization, and is suitable for acquiring the uniform thin film). However, in order to prevent the variable compound from reacting by external ultraviolet light, a series of film forming processes were performed under a yellow lamp. It was separately verified that the uniform thin film was acquired although the variable compound was added at the rate, by previously considering film thickness distribution and the like. The undiluted solution and the mixed solution were coated on the glass substrate by a spin coating method with a film thickness of 100 nm and temporarily dried at a temperature of 80° C. for 10 minutes, and then, imidized for 10 minutes after changing a temperature within the range of 180° C. to 250° C. The result of measuring an imidization ratio of the acquired thin film (represented by percentage) was shown in Table 1. First, considering a relationship between an imidized temperature and an imidization ratio, with respect to an additive amount of 0 wt % as a Comparative Example, the imidization was nearly not performed by 9.5% at a temperature of 180° C., but was rapidly performed from 200° C. or more, and the imidization ratio was 56.7% at a temperature of 250° C. In addition, although not shown in the Table, the imidization ratio was 57.1% at 350° C. and reached to the upper limit at 250° C. In contrast, in the imidization ratio of the mixture, the imidization ratio was improved at all temperatures as the additive amount increased. However, when the imidization temperature was 180° C., the imidization ratio was only about 10% and although the additive concentration was high, the imidization was not sufficiently acquired. Meanwhile, considering a temperature at which the imidization of 50% or more could be achieved, the imidization temperature of 230° C. or more was required for the additive of 0 wt %, but when the additive was added, the temperature of 220° C. or more was sufficient and particularly, when the additive of 40 wt % or more was added, the temperature of 210° C. was sufficient. As described above, as the additive amount of the variable compound increased, the imidization was efficiently performed. It was checked whether polyamic acid which was the raw material was not dissolved in the additive amount of 100%, that is, the variable compound itself, but the polymer of Example 1 was not dissolved. However, this does not mean that another polymer may not be dissolved.

As described above, it was confirmed that the imidization ratio was improved by using the solvent for forming an alignment film including the variable compound according to the embodiment of the present invention.

TABLE 1

| Additive | | | | | | | |
|---|---|---|---|---|---|---|---|
| amount | Imidization temperature (° C.) | | | | | | |
| (wt %) | 180 | 200 | 210 | 220 | 230 | 240 | 250 |
| 0 | 9.5 | 30.7 | 43.8 | 48.9 | 51.7 | 54.9 | 56.7 |
| 10 | 9.7 | 31.3 | 43.9 | 52.2 | 53.3 | 57.4 | 57.6 |
| 20 | 10.2 | 32.1 | 47.4 | 53.3 | 57.0 | 60.4 | 61.6 |
| 30 | 10.9 | 32.9 | 47.1 | 54.4 | 57.4 | 58.6 | 60.8 |
| 40 | 11.1 | 34.9 | 50.4 | 57.4 | 59.0 | 62.2 | 64.2 |
| 50 | 11.9 | 37.6 | 54.5 | 59.8 | 62.5 | 64.3 | 69.6 |
| 60 | 12.4 | 38.5 | 58.9 | 64.5 | 69.6 | 72.1 | 73.1 |
| 70 | 12.9 | 40.9 | 58.1 | 69.4 | 74.5 | 75.2 | 76.8 |
| 80 | 13.9 | 41.6 | 60.0 | 68.8 | 75.5 | 78.0 | 79.7 |
| 90 | 14.1 | 43.0 | 61.8 | 73.1 | 77.8 | 80.9 | 82.3 |

### Example 2

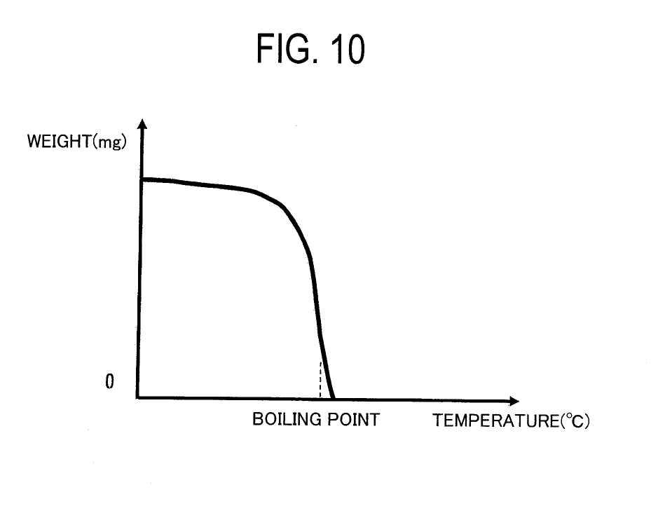

Subsequently, it was examined whether the boiling point of the solvent for forming an alignment film in order to form the alignment film including the polymer for a liquid crystal display according to the embodiment of the present invention was changed before and after the ultraviolet light was irradiated from the outside. The boiling point of the solvent was measured by using a thermogravimeter (although not particularly limited, the measurement was also performed under a yellow room). FIG. 10 shows a graph of variation in a weight in the thermogravity measurement. When a certain amount of solvent was put and the temperature increased for 1 minute by 1° C., a temperature at which the weight decreased by 95% as compared with the initial time was set as a boiling point before light irradiation. In order to irradiate the ultraviolet light to the solvent, a predetermined amount of sample solvent was put in a thermogravimetic container and the ultraviolet light of a wavelength of 330 nm was convergent-irradiated (the entire amount of light of 5 mJ/cm² in a collecting part) by combining an ultraviolet lamp (a high-pressure mercury lamp) and an interference filter while being maintained at 10° C. with a chiller. As described above, the boiling point after the light irradiation was measured in the same order by being set in the thermogravimeter. The result thereof was shown in Table 2. In Table 2, the solvent of the additive amount of 0 wt % as Comparative Example was not changed in the boiling point of 198° C. although the ultraviolet light is irradiated, but as the additive amount increased, the boiling point before the light irradiation increased. However, all of the boiling points after the light irradiation were lowered as compared with the boiling point before the light irradiation. Among them, as shown in Example 1, when the additive of 40 wt % or more was added, the imidization of 50% or more may be achieved even at 210° C., but in this case, the boiling point of the solvent before the light irradiation was 220° C. or more.

TABLE 2

| Additive | Boiling point | |
|---|---|---|
| amount | (° C.) | |
| (wt %) | Before | After |
| 0 | 198 | 198 |
| 10 | 202 | 198 |
| 20 | 210 | 199 |
| 30 | 218 | 200 |
| 40 | 221 | 199 |
| 50 | 226 | 199 |
| 60 | 228 | 200 |
| 70 | 231 | 199 |
| 80 | 232 | 198 |
| 90 | 230 | 199 |

Separately, a test piece of the thin film was prepared, inserted into a specific collecting tube capable of irradiating the ultraviolet light from the outside, and mounted on a gas chromatogram-mass spectroscope, and then, the temperature of the sample slowly increased, thereby tracking a molecular weight of a generated gas component. As a result, the component corresponding to a general solvent (N-methyl-2-pyrrolidone=99.12, γ-butyrolactone=86.09) used in the thin-film formation was detected at the heating temperature of about 190° C. and in the case where the ultraviolet light was not irradiated, a component having a molecular weight different from the component was generated at 228° C. and the component corresponded to a molecular weight component (148.20) of the solvent for forming an alignment film. Subsequently, when a separate thin-film sample was prepared, heated, and irradiated with the ultraviolet light during the heating, a completely new molecular weight component was generated soon after the ultraviolet irradiation of room temperature or more and corresponded to ethylene (28.05). When the temperature further increased, the general solvent component was detected at about 190° C. and a component having a larger molecular weight was included therein and corresponded to acetophenone (120.15). Although the heating was performed up to a higher temperature, the molecular weight component (148.20) could not be found. Accordingly, it was confirmed that the solvent for forming an alignment film was cleaved to fragments having smaller molecular weights by the ultraviolet irradiation.

As described above, in the solvent for forming an alignment film including the variable compound according to the embodiment of the present invention, the boiling point before the chemical structural change was higher than boiling point after the chemical structural change and particularly, in the case of the boiling point of 220° C. or more, the imidization ratio was improved.

### Example 3

Subsequently, alignment characteristics will be described when the alignment film was formed by using the solvent for forming an alignment film in order to form the alignment film including the polymer for a liquid crystal display according to the embodiment of the present invention.

A synthetic quartz substrate was used as the substrate and the undiluted solution of the raw material of the alignment film and the mixed solution with the composition as shown in Table 1 were coated, temporarily-dried, imidization-sintered in the same order as Example 1 (not particularly limited, but the processes also were performed under a yellow room). That is, the undiluted solution and the mixed solution were coated on the glass substrate by a spin coating method with a film thickness of 100 nm and temporarily dried at a temperature of 80° C. for 10 minutes, and then, imidized for 10 minutes after changing a temperature within 210° C., 230° C., and 250° C. In the imidized thin film, the ultraviolet light was uniformly irradiated (the entire amount of light of 5 mJ/cm²) to the entire film by the ultraviolet lamp (wavelength of 330 nm) at a substrate temperature of 230° C. as shown Example 2. Herein, for comparison, the ultraviolet light was not irradiated to the additive amount of 0 wt % of the variable compound. Particularly, when an absorption spectrum of the thin film was evaluated in this state, light was absorbed in a wavelength of 300 nm or less and a peak was shown in 262 nm. Subsequently, the polarized ultraviolet light (main wavelength of 280 nm, degree of polarization of 80:1) was uniformly irradiated (the entire amount of light of 5 mJ/cm²) to the entire film with a separate ultraviolet lamp (low-pressure mercury lamp), a wire grid polarizer, and an interference filter by returning the substrate temperature to room temperature. (The film finishing the above processing was referred to as a “film in which the polarized ultraviolet irradiation for the photoalignment was completed”) and thereafter, the substrate was heated at 230° C. for 20 minutes in the atmosphere.

In order to check a change in a molecular composition before and after the photoalignment with respect to the film in which the photoalignment processing was completed, a molecular weight of a molecular fragment in the film was examined by a liquid chromatography-mass analysis and as a result, a bismaleimide derivative (2,2′-dimethyl-1,1′-(4,4′-phenylene)bismaleimide, molecular weight of 296) in which a cyclobutane ring of a polymer chain of Chemical Formula 7 was cleaved or a component (molecular weight of 593, 625, and 657) considered to be derived from a dimer thereof was detected. Considering the molecular weights, when samples of which a heating time was changed before the ultraviolet irradiation, immediately after the ultraviolet irradiation, and after the ultraviolet irradiation were prepared again and an amount of the fragment component of each sample was tracked, the amount thereof was not detected from the sample before the ultraviolet irradiation, a maximum amount of the fragment component was detected immediately after the ultraviolet irradiation, and the component thereof was reduced together with the increase of the heating time.

TABLE 3

| | Film order parameter | | | Surface order parameter | | |
|---|---|---|---|---|---|---|
| Additive | Imidization temperature | | | Imidization temperature | | |
| amount | (° C.) | | | (° C.) | | |
| (wt %) | 210 | 230 | 250 | 210 | 230 | 250 |
| 0 (None) | 0.182 | 0.398 | 0.569 | 0.321 | 0.546 | 0.660 |
| 0 | 0.183 | 0.400 | 0.579 | 0.323 | 0.544 | 0.663 |
| 10 | 0.198 | 0.466 | 0.614 | 0.335 | 0.587 | 0.694 |
| 20 | 0.264 | 0.565 | 0.683 | 0.435 | 0.674 | 0.754 |
| 30 | 0.273 | 0.588 | 0.654 | 0.410 | 0.728 | 0.750 |
| 40 | 0.368 | 0.611 | 0.739 | 0.492 | 0.719 | 0.773 |
| 50 | 0.500 | 0.720 | 0.799 | 0.613 | 0.768 | 0.858 |
| 60 | 0.613 | 0.783 | 0.841 | 0.744 | 0.840 | 0.855 |
| 70 | 0.603 | 0.814 | 0.815 | 0.737 | 0.868 | 0.862 |
| 80 | 0.644 | 0.806 | 0.834 | 0.747 | 0.846 | 0.890 |
| 90 | 0.704 | 0.850 | 0.877 | 0.761 | 0.907 | 0.920 |

A film order parameter and a surface order parameter of the alignment film acquired as described above were measured. The result thereof was shown in Table 3. First, on the whole, as the imidization temperature increased, the film order parameter and the surface order parameter tended to have higher values and under the same condition, the film order parameter had a higher value than the surface order parameter. Subsequently, with respect to the additive amount of 0 wt %, the order parameters had the same value regardless of the light irradiation of the ultraviolet lamp (wavelength of 330 nm) and the used polyimide did not react with the ultraviolet light of the wavelength. Subsequently, in the case where the variable compound was added, the order parameter increased together with the increase of the additive amount and the order parameters of the acquired alignment film increased by using the solvent for forming the alignment film according to the embodiment of the present invention. In particular, when the variable compound was not added, the heating of 250° C. was required so that the order parameter becomes 0.5 or more. However, when using the solvent of the present invention, although when the additive amount was 20 wt % or more, the heating temperature was 230° C., and when the additive amount was 50 wt % or more, the heating temperature was 210° C., the order parameter was 0.5 or more and the temperature of the heating process was efficiently reduced. (For more confirmation, the non-polarized ultraviolet light of the same wavelength was irradiated, but in this case, the order parameter was 0 and no alignment was made). As described above, the solvent for forming the alignment film including the variable compound of the present invention may change the chemical structure by coating the solution for an alignment film on the substrate and irradiating the light from the outside and the quality of the acquired alignment film was improved.

### Example 4

Subsequently, anchoring characteristics will be described when the alignment film was formed by using the solvent for forming an alignment film in order to form the alignment film including the polymer for a liquid crystal display according to the embodiment of the present invention.

The glass substrate was used as the substrate, the alignment film was formed by the composition shown in Table 1 in the order as shown Example 3, the film in which the polarized ultraviolet irradiation for the photoalignment was completed was acquired in the same order, and the substrate was heated at 230° C. for 20 minutes in the atmosphere. By using two substrates as one set, the liquid crystal cell for evaluating an anchoring characteristic was manufactured to be parallel to the alignment processing direction.

TABLE 4

| Additive | Imidization | | |
|---|---|---|---|
| amount | temperature (° C.) | | |
| (wt %) | 210 | 230 | 250 |
| 0 (None) | 1.28 | 3.49 | 5.52 |
| 0 | 1.29 | 3.46 | 5.57 |
| 10 | 1.33 | 4.09 | 6.05 |
| 20 | 2.04 | 5.66 | 6.94 |
| 30 | 2.18 | 6.42 | 7.05 |
| 40 | 2.73 | 6.67 | 7.19 |
| 50 | 4.51 | 7.11 | 8.21 |
| 60 | 6.73 | 8.13 | 8.27 |
| 70 | 6.80 | 8.21 | 8.37 |
| 80 | 6.87 | 8.41 | 8.86 |
| 90 | 7.12 | 8.65 | 8.98 |

Unit: mJ/m²

The anchoring of the liquid crystal cell acquired as described above was measured. The result thereof was shown in Table 4. First, on the whole, as the imidization temperature increased, the anchoring strength tended to be high. Subsequently, with respect to the additive amount of 0 wt %, the anchoring strength had substantially the same value regardless of the light irradiation of the ultraviolet lamp (wavelength of 330 nm) and the used polyimide did not react with the ultraviolet light of the wavelength. Subsequently, in the case where the variable compound was added, the anchoring strength increased together with the increase of the additive amount and the anchoring strength of the acquired alignment film increased by using the solvent for forming the alignment film according to the embodiment of the present invention. In particular, when the variable compound was not added, the heating of 230° C. or more was required so that the anchoring strength is 3 mJ/m² or more. However, by using the solvent of the present invention, although when the additive amount was 50 wt % or more, the heating temperature was 210° C., the anchoring strength was 3 mJ/m2 or more and the temperature of the heating process was efficiently reduced.

Further, by using the liquid crystal cell, as a result of measuring pretilt angles in the liquid crystal, all the pretilt angles were 0 degree and the regulatory force of aligning the liquid crystal was given in the film plane.

Subsequently, when the angle of the polarized ultraviolet irradiation for the photoalignment was changed, the pretilt angles in the liquid crystal cell acquired from the alignment film were measured. In detail, generally, the ultraviolet light is irradiated in a perpendicular direction to the substrate surface (a polarization axis is in the film plane), but herein, the ultraviolet light was irradiated by inclining the substrate. The acquired pretilt angle of the liquid crystal cell configured by the alignment film was substantially the same as the inclined angle of the substrate and the regulatory force of aligning the liquid crystal in a line direction was given in the film plane. In particular, when the pretilt angle was 30 degrees or more, the liquid crystal cell was substantially close to a vertical alignment state in the initial time and as a result, when the polarizer having mutually orthogonal polarization axes was inserted between the upper and the lower of the liquid crystal cell, it is possible to form a dark viewing state without significantly recognizing the polarization axis direction of the polarizer.

As described above, the solvent for forming the alignment film including the variable compound of the present invention may change the chemical structure by coating the solution for an alignment film on the substrate and irradiating the light from the outside and the quality of the alignment film was improved when the acquired alignment film was assembled with the liquid crystal cell.

### Example 5

Subsequently, by using the alignment film shown in Example 4, the IPS mode liquid crystal display element shown in FIG. 7 was fabricated and the evaluation result of the transmittance thereof was shown.

The liquid crystal panel was fabricated by the same process as a general fabricating process, except that the solvent for forming an alignment film according to the embodiment of the present invention was used as an alignment film material. For example, in a representative manufacturing method of the IPS mode liquid crystal display, the cell was fabricated by bonding the active matrix substrate 106 and the opposed substrate 107 which were alignment-processed in advance to seal the liquid crystal material, but in this case, an initial alignment direction of the alignment film 606 of the active matrix substrate 106 is substantially parallel to an initial alignment direction of the alignment film 705 of the opposed substrate 107. Further, as the sealed liquid crystal material, for example, a nematic liquid crystal composition A which has a positive dielectric anisotropy Δ∈ of 10.2 (1 kHz, 20° C.), a refraction ratio anisotropy of 0.075 (wavelength of 590 nm, 20° C.), a twist elastic constant K₂ of 7.0 pN, a nematic-isotropic phase transition temperature T(N—I) of about 76° C., and a specific resistance of 1×10+13 Ωcm is used. Further, the active matrix substrate 106 and the opposed substrate 107 are bonded with each other so that a thickness (cell gap) of the liquid crystal layer LC is substantially the same as a height of the columnar spacer 110, for example, 4.2 μm. A retardation Δn·d of the liquid crystal panel 101 fabricated by the condition is about 0.31 μm. The retardation Δn·d is preferably in the range of 0.2 μm≦Δn·d≦0.5 μm and if the retardation exceeds the range, there is a problem in that a white display is colored. When the liquid crystal material is sealed by bonding the active matrix substrate 106 and the opposed substrate 107, for example, the polarizers 109a and 109b are bonded with each other by cutting and removing unnecessary portions (margin portions) of outer circumferences of the glass substrates 601 and 701. When the polarizers 109a and 109b are bonded with each other, the polarization transmission axis of one polarizer is substantially parallel to the initial alignment direction of the alignment film 606 of the active matrix substrate 106 and the alignment film 705 of the opposed substrate 107 and the polarization transmission axis of the other polarizer is perpendicular to the initial alignment direction. Thereafter, the liquid crystal display including the liquid crystal panel 101 of Example 5 is acquired by connecting and modularizing the first driving circuit 102, the second driving circuit 103, the control circuit 104, the backlight 105, and the like. Meanwhile, the liquid crystal panel 101 of Example 5 has a normally close characteristic in which the liquid crystal panel was darkly displayed (displayed in low luminance) when a potential difference between the pixel electrode PX and the common electrode CT is small and brightly displayed (displayed in high luminance) when a potential difference between the pixel electrode PX and the common electrode CT is large. Another mode of liquid crystal display is generally manufactured by each method to be darkly and brightly displayed.

TABLE 5

| Additive | Imidization | | |
|---|---|---|---|
| amount | temperature (° C.) | | |
| (wt %) | 210 | 230 | 250 |
| 0 (None) | 78.7 | 34.8 | 22.0 |
| 0 | 79.5 | 35.8 | 22.4 |
| 10 | 76.9 | 29.3 | 20.8 |
| 20 | 58.4 | 21.6 | 18.8 |
| 30 | 56.9 | 20.2 | 18.9 |
| 40 | 46.0 | 19.5 | 18.6 |
| 50 | 27.5 | 18.4 | 17.1 |
| 60 | 19.2 | 17.0 | 16.8 |
| 70 | 19.4 | 16.9 | 16.8 |
| 80 | 18.6 | 16.8 | 16.2 |
| 90 | 18.6 | 16.7 | 16.1 |

Unit: minute

An afterimage time of the liquid crystal display element acquired as described above was measured. The result thereof was shown in Table 5. First, on the whole, as the imidization temperature increased, the afterimage time tended to be short. Subsequently, with respect to the additive amount of 0 wt %, the afterimage time had substantially the same value regardless of the light irradiation of the ultraviolet lamp (wavelength of 330 nm) and the ultraviolet irradiation of the wavelength did not influence the performance of the liquid crystal display element. Subsequently, in the case where the variable compound was added, the afterimage time decreased according to the increase of the additive amount and the afterimage time was reduced by using the solvent for forming the alignment film according to the embodiment of the present invention.

As described above, the solvent for forming the alignment film including the variable compound of the present invention may change the chemical structure by coating the solution for an alignment film on the substrate and irradiating the light from the outside and the performance was improved when the acquired alignment film was assembled with the IPS mode liquid crystal display element.

### Example 6

Subsequently, by using the alignment film shown in Example 4, the FFS mode liquid crystal display element shown in FIG. 8 was fabricated and the evaluation result of the light transmittance thereof will be described. The element structure of the FFS mode is similar to that of the IPS mode and the pixel electrode PX and the common electrode CL are formed only one side of the upper and lower base substrates and the liquid crystal rotates in the plane according to an electric field therebetween. Accordingly, the initial alignment state in which the electric field is not applied is also similar to that of the IPS mode and may be similar to the alignment direction to be performed in the alignment films 606 (and 705) and the liquid crystal having the positive dielectric anisotropy A may also be used.

TABLE 6

| Additive | Imidization | | |
|---|---|---|---|
| amount | temperature (° C.) | | |
| (wt %) | 210 | 230 | 250 |
| 0 (None) | 107.5 | 47.2 | 29.7 |
| 0 | 105.3 | 46.9 | 29.8 |
| 10 | 104.4 | 40.2 | 27.9 |
| 20 | 77.5 | 29.1 | 24.9 |
| 30 | 74.5 | 26.1 | 24.9 |
| 40 | 60.8 | 26.0 | 24.6 |
| 50 | 35.6 | 24.6 | 23.1 |
| 60 | 25.2 | 23.1 | 22.7 |
| 70 | 25.3 | 22.8 | 22.3 |
| 80 | 25.2 | 22.1 | 22.3 |
| 90 | 24.8 | 22.4 | 22.0 |

Unit: minute

An afterimage time of the liquid crystal display element acquired as described above was measured. The result thereof was shown in Table 6. First, on the whole, as the imidization temperature increased, the afterimage time tended to be short. Subsequently, with respect to the additive amount of 0 wt %, the afterimage time substantially had the same value regardless of the light irradiation of the ultraviolet lamp (wavelength of 330 nm) and the ultraviolet irradiation of the wavelength did not influence the performance of the liquid crystal display element. Subsequently, in the case where the variable compound was added, the afterimage time decreased according to the increase of the additive amount and the afterimage time was reduced by using the solvent for forming the alignment film according to the embodiment of the present invention.

As described above, the solvent for forming the alignment film including the variable compound of the present invention may change the chemical structure by coating the solution for an alignment film on the substrate and irradiating the light from the outside and the performance was improved when the acquired alignment film was assembled with the FFS mode liquid crystal display element.

### Example 7

Subsequently, by using the alignment film shown in Example 4, the VA mode liquid crystal display element shown in FIG. 9 was fabricated and the evaluation result of the light transmittance thereof will be described. Unlike the IPS mode or the FFS mode, in the VA mode, the pixel electrode PX and the common electrode CL are formed on the upper and lower base substrate, the liquid crystal material for a VA mode having negative dielectric anisotropy Δ∈ is used, in the initial alignment state in which the electric field is not applied, the liquid crystal molecules should be processed so as to be aligned nearly perpendicular to the substrate surface. Accordingly, it is difficult to use the general rubbing. Herein, referring to “Patterned photoalignment for vertically aligned LCDs” (P. Gass, H. Stevenson, R. Bay, H. Walton, N. Smith, S. Terashita, and M. Tillin, SHARP TECHNICAL JOURNAL No. 85 (2003) 24-29), the photoalignment processing was performed by irradiation the polarized ultraviolet light from the inclined direction.

TABLE 7

| Additive | Imidization | | |
|---|---|---|---|
| amount | temperature (° C.) | | |
| (wt %) | 210 | 230 | 250 |
| 0 (None) | 185.4 | 83.2 | 52.5 |
| 0 | 184.1 | 82.6 | 50.8 |
| 10 | 178.9 | 70.8 | 49.2 |
| 20 | 140.0 | 51.1 | 44.5 |
| 30 | 131.3 | 46.3 | 44.2 |
| 40 | 106.1 | 45.9 | 43.3 |
| 50 | 61.8 | 43.4 | 39.6 |
| 60 | 44.2 | 40.5 | 40.1 |
| 70 | 44.8 | 39.5 | 39.3 |
| 80 | 43.9 | 39.4 | 38.0 |
| 90 | 44.0 | 38.3 | 38.9 |

Unit: minute

An afterimage time of the liquid crystal display element acquired as described above was measured. The result thereof was shown in Table 7. First, on the whole, as the imidization temperature increased, the afterimage time tended to be short. Subsequently, with respect to the additive amount of 0 wt %, the afterimage time had substantially the same value regardless of the light irradiation of the ultraviolet lamp (wavelength of 330 nm) and the ultraviolet irradiation of the wavelength did not influence the performance of the liquid crystal display element. Subsequently, in the case where the variable compound was added, the afterimage time decreased according to the increase of the additive amount and the afterimage time was reduced by using the solvent for forming the alignment film according to the embodiment of the present invention.

As described above, the solvent for forming the alignment film including the variable compound of the present invention may change the chemical structure by coating the solution for an alignment film on the substrate and irradiating the light from the outside and the performance was improved when the acquired alignment film was assembled with the VA mode liquid crystal display element.

### Example 8

Subsequently, in the case where the solvent for forming an alignment film having a separate chemical structure is used, a result of examining a characteristic thereof will be described.

Previously, the use of the solvent for forming an alignment film of Chemical Formula 6 was reviewed, but hereinafter, the solvents for forming an alignment film of the following Chemical Formulas 8 and 9 were used. In addition, the alignment film component, the main solvent thereof, and the forming process of the alignment film thereof were the same as Example 1. The evaluation method of the imidization ratio and the like were the same as Example 1.

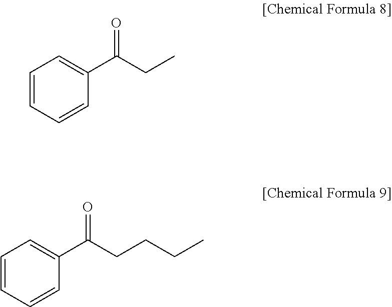

Table 8 shows a result (represented by percentage) of measuring the imidization ratio of a thin film acquired by Chemical Formula 8. Like Example 1, as compared with the case where the solvent for forming an alignment film of the present invention was not used, as the additive amount increased, the imidization ratio was improved, but there is little difference in the imidization ratio at the imidization temperature of 230° C. or more. This is assumed because the separately measured boiling point of the compound was about 220° C., such that the compound is nearly evaporated at a high temperature and there was no effect of the adding.

TABLE 8

| Additive | | | | | | | |
|---|---|---|---|---|---|---|---|
| amount | Imidization temperature (° C.) | | | | | | |
| (wt %) | 180 | 200 | 210 | 220 | 230 | 240 | 250 |
| 0 | 9.5 | 30.7 | 43.8 | 48.9 | 51.7 | 54.9 | 56.7 |
| 10 | 9.9 | 30.6 | 43.7 | 49.1 | 51.2 | 54.4 | 56.5 |
| 20 | 10.3 | 31.4 | 44.0 | 48.6 | 51.5 | 55.5 | 57.1 |
| 30 | 10.6 | 31.3 | 43.7 | 48.9 | 52.1 | 54.7 | 56.5 |
| 40 | 10.8 | 31.4 | 44.2 | 48.6 | 52.1 | 54.8 | 56.3 |
| 50 | 11.1 | 32.0 | 44.1 | 49.2 | 51.6 | 54.8 | 56.5 |
| 60 | 11.7 | 31.8 | 44.1 | 48.6 | 51.9 | 54.7 | 56.5 |
| 70 | 11.9 | 32.2 | 44.5 | 48.5 | 51.3 | 54.5 | 56.6 |
| 80 | 12.1 | 32.7 | 44.5 | 49.1 | 51.4 | 55.3 | 57.3 |
| 90 | 12.6 | 32.5 | 43.8 | 49.1 | 51.8 | 54.8 | 57.4 |

Table 9 shows a result (represented by percentage) of measuring the imidization ratio of a thin film acquired by Chemical Formula 9. Like Example 1, as compared with case where the solvent for forming an alignment film of the present invention was not used, as the additive amount increased, the imidization ratio was improved, but in the case of using the material of Chemical Formula 6 of Example 1, the improved effect became higher and the maximum imidization ratio of 83% was acquired. This is assumed because the separately measured boiling point of the compound was about 245° C., such that the compound as the solvent remains until being imidized-sintered up to a higher temperature.

TABLE 9

| Additive | | | | | | | |
|---|---|---|---|---|---|---|---|
| amount | Imidization temperature (° C.) | | | | | | |
| (wt %) | 180 | 200 | 210 | 220 | 230 | 240 | 250 |
| 0 | 9.5 | 30.7 | 43.8 | 48.9 | 51.7 | 54.9 | 56.7 |
| 10 | 10.1 | 32.0 | 46.0 | 51.4 | 54.9 | 58.3 | 59.2 |
| 20 | 10.6 | 33.2 | 47.4 | 54.2 | 57.6 | 61.3 | 62.0 |
| 30 | 11.3 | 35.1 | 50.1 | 57.2 | 60.5 | 64.0 | 64.9 |
| 40 | 11.9 | 36.4 | 52.2 | 60.3 | 63.2 | 66.7 | 68.8 |
| 50 | 12.4 | 37.9 | 53.6 | 62.5 | 66.0 | 69.3 | 70.9 |
| 60 | 12.9 | 39.0 | 56.3 | 65.1 | 68.8 | 72.6 | 74.1 |
| 70 | 13.4 | 40.5 | 57.6 | 67.4 | 71.9 | 75.6 | 77.5 |
| 80 | 13.9 | 42.2 | 59.7 | 70.6 | 75.3 | 79.5 | 79.7 |
| 90 | 14.4 | 43.8 | 61.4 | 73.1 | 78.3 | 82.4 | 83.2 |

As described above, the imidization ratio was improved by using the solvent for forming an alignment film including the variable compound according to the embodiment of the present invention.

Subsequently, as shown in Example 4, as a result of checking the alignment performance of the liquid crystal molecules by assembling the liquid crystal cell for evaluating the anchoring strength, the uniform alignment of the liquid crystal was checked by observation through a polarized microscope in the liquid crystal cell configured by the alignment film acquired from the solvent for forming an alignment film.

Subsequently, while the additive amount was 70% and the imidization sintering temperature was 210° C., as shown in Examples 5 to 7, three kinds of liquid crystal display elements were formed by using the solvent, and the liquid crystal display element was also fabricated without the additive amount. Thereafter, when comparing the afterimage times thereof, the afterimage times decreased and the afterimage characteristics were improved as compared with the liquid crystal display element fabricated without the additive amount.

### Example 9

Subsequently, in the case where the solvent for forming an alignment film having a separate chemical structure is used, a result examining a characteristic thereof will be described.

Herein, the solvent for forming an alignment film of the following Chemical Formula 10 was used. In addition, the alignment film component, the main solvent thereof, and the forming process of the alignment film thereof were the same as Example 1. The evaluation method of the imidization ratio and the like were the same as Example 1.

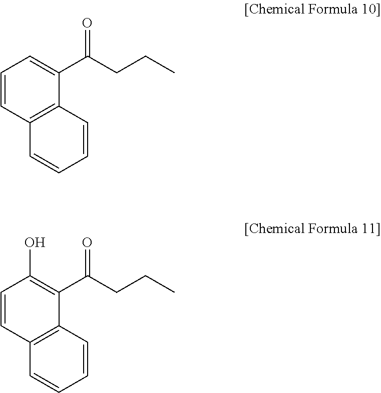

Table 10 shows a result (represented by percentage) of measuring the imidization ratio of a thin film acquired by Chemical Formula 10. Like Example 1, as compared with the case where the solvent for forming an alignment film of the present invention was not used, as the additive amount increased, the imidization ratio was improved, but in the case where the material of Chemical Formula 6 of Example 1 was used, the improved effect became higher and the maximum imidization ratio of 77% was acquired even in the additive amount of 60%. When an ultraviolet-visible absorption spectrum of the thin film was measured at the point of time when the imidization-sintering was completed, a peak was shown around 330 to 340 nm and extended to about 360 nm. When the solvent for forming the alignment film was not added, the absorption peak was not shown in 300 nm and the absorption was derived from the solvent for forming the alignment film. When the photoalignment processing was performed on the thin film in the same order as the method shown in Example 3, the absorption peak decreased by about 10%, but the strong absorption peak remained in the ultraviolet area. Further, when the anchoring strength of the alignment film surface was measured by the method shown in Example 4 by using the photoalignment-processed alignment film, the anchoring strength was 3 mJ/m² or more in the additive amount of 20 to 50%, but the anchoring strength was reduced by 2.4 to 2.8 mJ/m² in the additive amount of 60%. Therefore, after the polarized ultraviolet irradiation for the photoalignment was completed, when in the heating and drying process in the atmosphere, the heating was performed at the same temperature in a decompressed chamber of 1 mmTorr or less, a good characteristic in which the anchoring strength was 3 mJ/m² or more was shown.

TABLE 10

| Additive | | | | | | | |
|---|---|---|---|---|---|---|---|
| amount | Imidization temperature (° C.) | | | | | | |
| (wt %) | 180 | 200 | 210 | 220 | 230 | 240 | 250 |
| 0 | 9.5 | 30.7 | 43.8 | 48.9 | 51.7 | 54.9 | 56.7 |
| 10 | 10.5 | 32.5 | 45.9 | 52.3 | 55.2 | 58.1 | 59.7 |
| 20 | 11.5 | 34.9 | 47.8 | 55.4 | 57.5 | 61.4 | 64.4 |
| 30 | 12.6 | 36.1 | 50.6 | 57.9 | 61.1 | 64.9 | 67.9 |
| 40 | 13.7 | 38.4 | 52.4 | 61.4 | 65.0 | 69.1 | 70.8 |
| 50 | 14.9 | 40.5 | 54.8 | 64.4 | 67.8 | 71.7 | 74.0 |
| 60 | 16.0 | 42.6 | 57.0 | 67.3 | 71.3 | 74.5 | 77.7 |

Table 11 shows a result (represented by percentage) of measuring the imidization ratio of a thin film acquired by Chemical Formula 11. Like Example 1, as compared with the case where the solvent for forming an alignment film of the present invention was not used, as the additive amount increased, the imidization ratio was improved, but in the case where the material of Chemical Formula 6 of Example 1 was used, the improved effect became higher and the maximum imidization ratio of 77% was acquired even in the additive amount of 60%. When an ultraviolet-visible absorption spectrum of the thin film was measured at the time of period when the imidization-sintering was completed, a peak was shown around 340 to 360 nm and extended to about 380 nm. In the case where the solvent for forming the alignment film was not added, the absorption peak was not shown in 300 nm and the absorption was derived from the solvent for forming the alignment film. When the photoalignment processing was performed on the thin film in the same order as the method shown in Example 3, the absorption peak decreased by about 10%, but the strong absorption peak remained in the ultraviolet area. Further, when the anchoring strength of the alignment film surface was measured by the method shown in Example 4 by using the photoalignment-processed alignment film, the anchoring strength was 3 mJ/m² or more in the additive amount of 20 to 50%, but the anchoring strength was reduced by 2.4 to 2.8 mJ/m2 in the additive amount of 60%. Therefore, after the polarized ultraviolet irradiation for the photoalignment was completed, when in the heating and drying process in the atmosphere, the heating was performed at the same temperature in a decompressed chamber of 1 mmTorr or less, a good characteristic in which the anchoring strength was 3 mJ/m² or more was shown.

TABLE 11

| Additive | | | | | | | |
|---|---|---|---|---|---|---|---|
| amount | Imidization temperature (° C.) | | | | | | |
| (wt %) | 180 | 200 | 210 | 220 | 230 | 240 | 250 |
| 0 | 9.5 | 30.7 | 43.8 | 48.9 | 51.7 | 54.9 | 56.7 |
| 10 | 10.5 | 32.9 | 46.3 | 51.8 | 54.7 | 58.3 | 59.7 |
| 20 | 11.6 | 34.6 | 48.1 | 55.4 | 57.9 | 61.8 | 64.2 |
| 30 | 12.8 | 36.6 | 50.5 | 57.8 | 61.0 | 64.9 | 66.9 |
| 40 | 13.8 | 38.5 | 52.5 | 61.5 | 63.9 | 69.1 | 70.5 |
| 50 | 14.9 | 40.0 | 54.6 | 64.4 | 67.9 | 71.3 | 73.9 |
| 60 | 15.7 | 42.1 | 57.4 | 67.0 | 70.3 | 74.6 | 77.5 |

Subsequently, as shown in Example 4, when the alignment performance of the liquid crystal molecules was checked by assembling the liquid crystal cell for evaluating the anchoring strength, the uniform alignment of the liquid crystal was observed through a polarized microscope in the liquid crystal cell configured by the alignment film acquired from the solvent for forming an alignment film.

Subsequently, while the additive amount was 70% and the imidization sintering temperature was 210° C., as shown in Examples 5 to 7, three kinds of liquid crystal display elements were formed by using the solvent and the liquid crystal display element was also fabricated without the additive amount. Thereafter, when comparing the afterimage times thereof, the afterimage times decreased and the afterimage characteristics were improved as compared with the liquid crystal display element fabricated without the additive amount.

### Example 10

Subsequently, in the case of using the solvent for forming an alignment film having a separate chemical structure, the result examining a characteristic thereof will be described.

In the solvent for photoalignment used in Example 9, the partial backbone remained in the alignment film and the characteristic absorbing the ultraviolet may be given, but the upper limit of the additive amount was 60% and when the additive amount further increased, the solubility was deteriorated when polyamic acid as a precursor of polyimide was dissolved and as a result, polyamic acid was precipitated. Therefore, as shown in Chemical Formulas 12 to 19, the solvent for forming an alignment film including halogen atoms in a part of the molecular backbone was selected and the solvent was a solvent for forming a mixed alignment film which adds the additive of 10 to maximal 40 wt % in the solvent of Example 9. Accordingly, although the substantial additive amount of the solvent of Example 9 increased up to 85%, the alignment film could be uniformly formed. As described above, since the additive amount of the solvent of Example 9 increased, the concentration of the ultraviolet absorption component remaining in the alignment film may finally increase and the ultraviolet film having higher efficiency than Example 9 may be acquired. Further, since the components derived from Chemical Formulas 12 to 19 did not remain in the final alignment film, the halogen atoms were not observed by an atom analysis of a film.

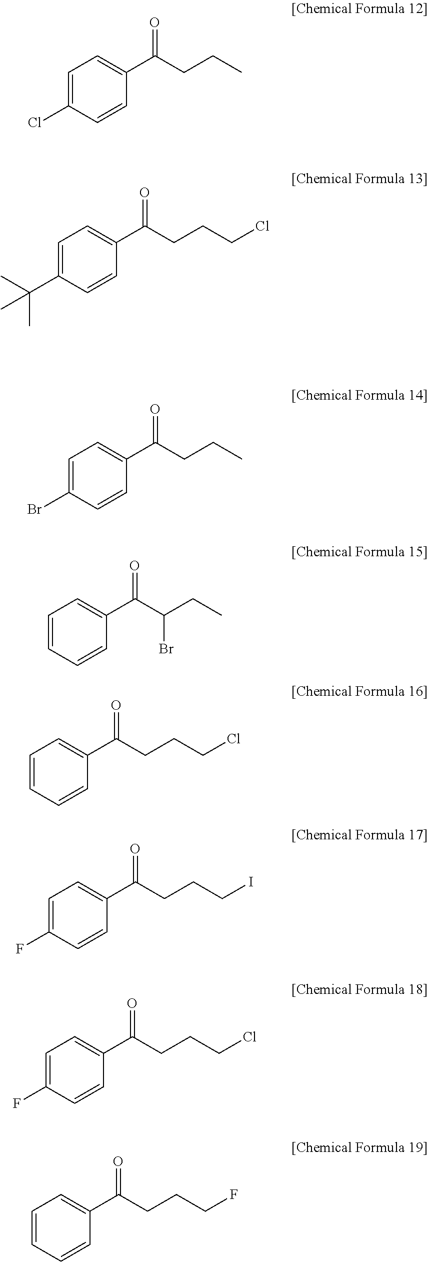

In order to verify an effect of the new solvents(?) for forming an alignment film, before and after irradiating the ultraviolet light for causing the structural change of the solvent for forming an alignment film, dielectric ratios of the thin film were measured in the following order. That is, the pixel electrode of 1 mmφ made of patterned ITO and a guard electrode surrounding the pixel electrode with an interval of 0.1 mm were formed on the quartz substrate and the polyimide thin film was formed thereon as described above. Herein, the imidization-sintering was performed and the ultraviolet irradiation was not performed. Subsequently, the opposed electrode was formed on the thin film by a gold deposition so as to cover from the pixel electrode to the guard electrode. As described above, the sample for measuring the dielectric ratio was prepared and then, the dielectric ratio was measured and the measured dielectric ratio was referred to as a dielectric ratio before the ultraviolet irradiation. Subsequently, the ultraviolet light for causing the structural change of the solvent for forming an alignment film was irradiated from the substrate to the thin film and then, similarly, the dielectric ratio was measured and the measured dielectric ratio was referred to as a dielectric ratio after the ultraviolet irradiation. Thereafter, when the dielectric ratio of only Chemical Formula 10 of Example 9 was checked, a specific dielectric ratio before the ultraviolet irradiation was 3.8 in the range of frequencies of 100 to 10 kHz, but a specific dielectric ratio after the ultraviolet irradiation was reduced by 3.2, such that polarity of the thin film was changed. Further, when adding Chemical Formulas 10 to 19 of Example 10, the specific dielectric ratio before the ultraviolet irradiation increased by 4.0 to 4.2 when Cl, Br, and I were added, the specific dielectric ratio decreased by 3.6 in the Chemical Formula 19 in which F was added, and the specific dielectric ratios were substantially the same as each other in Chemical Formulas 17 and 18 in which both Cl, Br, and I and F were added. In contrast, all the specific dielectric ratios after the ultraviolet irradiation decreased by 3.2 and the polarity of the thin film was changed. When the solvent for forming an alignment film has high polarity, it is advantageous in that the solubility of polyamic acid as the precursor of polyimide increases. However, in order to increase the polarity of a general solvent, the boiling point of the solvent also increases, such that a high heating temperature or a long heating time for removing the solvent is required and in some cases, the solvent may not be used due to heat resistance of other members such as the TFT portion or color filter of the liquid crystal display. Accordingly, by using the solvent in which the polarity is changed as in the present invention, the solvent which can be easily removed may be provided.

Subsequently, as shown in Example 4, when the alignment performance of the liquid crystal molecules was checked by assembling the liquid crystal cell for evaluating the anchoring strength, the uniform alignment of the liquid crystal was observed through a polarized microscope in the liquid crystal cell configured by the alignment film acquired from the solvent for forming an alignment film.

What is claimed is:

1. A solvent for forming an alignment film including a polymer for a liquid crystal display, comprising:
a variable compound capable of changing a chemical structure during a film-forming process of coating the polymer and forming the alignment film.

2. The solvent for forming an alignment film according to claim 1, wherein the polymer of the alignment film includes polyimide and a variable compound capable of changing a chemical structure for evaporating after imidizing and sintering, which is in a liquid state during a film-forming process of coating polyamic acid which is a precursor of polyimide, imidizing, and sintering.

3. The solvent for forming an alignment film according to claim 2, wherein in the variable compound, a boiling point before changing the chemical structure for evaporation is higher than a boiling point after changing the chemical structure.

4. The solvent for forming an alignment film according to claim 3, wherein in the variable compound, the boiling point before changing the chemical structure for evaporation is equal to or higher than 220° C.

5. The solvent for forming an alignment film according to claim 3, wherein in the variable compound, the boiling point after changing the chemical structure for evaporation is room temperature or more and 220° C. or less.

6. The solvent for forming an alignment film according to claim 2, wherein in the variable compound, a molecular weight before changing the chemical structure for evaporation is higher than a molecular weight after changing the chemical structure.

7. The solvent for forming an alignment film according to claim 2, wherein in the variable compound, polarities before and after changing the chemical structure for evaporation are different.

8. The solvent for forming an alignment film according to claim 1, wherein the variable compound includes a chemical structure represented by Chemical Formula 1: 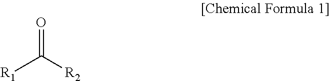
wherein, R₁ is an aromatic carbon compound and R₂ is an aliphatic carbon compound.

9. The solvent for forming an alignment film according to claim 8, wherein in the variable compound, R₁ is represented by Chemical Formula 2 or 3 of the chemical structure represented by Chemical Formula 1: 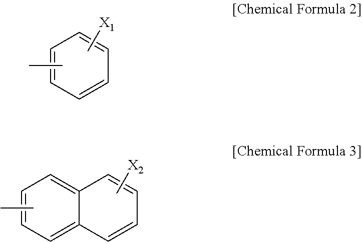
wherein, X₁ or X₂ is a substituent group.

10. The solvent for forming an alignment film according to claim 8, wherein in the variable compound, R₂ is represented by Chemical Formula 4 of the chemical structure represented by Chemical Formula 1: 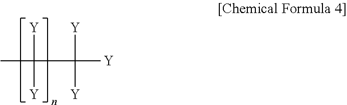
wherein, Y is any one of —H, —F, —Cl, —Br, and —I and in all substituent sites, Y is not be the same. In addition, n is an integer of 1 to 3.

11. The solvent for forming an alignment film according to claim 1, wherein the chemical structure of the variable compound is changed by coating an alignment film raw solution on the substrate, forming a thin film, and then irradiating light from the outside.

12. The solvent for forming an alignment film according to claim 11, wherein the chemical structure of the variable compound is changed by irradiating light having a wavelength of 400 nm or less from the outside.

13. The solvent for forming an alignment film according to claim 1, wherein the solvent is a solvent for forming polyimide forming an alignment film having an alignment regulatory force of the liquid crystal after evaporating the solvent for forming the alignment film.

14. The solvent for forming an alignment film according to claim 13, wherein the solvent is a solvent for forming polyimide forming an alignment film having an alignment regulatory force of the liquid crystal by a polarized light.

15. The solvent for forming an alignment film according to claim 14, wherein the solvent is a solvent for forming polyimide forming an alignment film having an alignment regulatory force of the liquid crystal by a polarized light having a wavelength different from the light changing the chemical structure of the variable compound.

16. The solvent for forming an alignment film according to claim 15, wherein the solvent is a solvent for forming polyimide forming an alignment film having an alignment regulatory force of the liquid crystal by a polarized light having a smaller wavelength than the light changing the chemical structure of the variable compound.

17. The solvent for forming an alignment film according to claim 14, wherein the solvent is a solvent for forming polyimide forming an alignment film having an alignment regulatory force of the liquid crystal by cutting a part of a main chain by the polarized light.

18. The solvent for forming an alignment film according to claim 1, wherein a part of the structure changing the chemical structure of the variable compound is a chemical structure remaining in the alignment film when the solvent for forming an alignment film is evaporated.

19. The solvent for forming an alignment film according to claim 18, wherein the chemical structure remaining in the alignment film is a chemical structure which is transparent with respect to visible light, but has absorption with respect to ultraviolet light.

20. A materials for an alignment film including the solvent for forming an alignment film according to claim 1 and polyamic acid which is a precursor of polyimide.

21. A method of manufacturing a liquid crystal display, in a process of forming an alignment film including a polymer for a liquid crystal display, comprising:
coating on a substrate a solution in which a polymer is dissolved in the solvent for forming an alignment film according to claim 1;
imidizing and sintering the coated polymer;
changing a chemical structure of the variable compound included in the solvent for forming an alignment film;
removing the solvent having the changed chemical structure; and
irradiating a polarized light for photoalignment on the remaining polymer thin film.

22. The method of manufacturing a liquid crystal display according to claim 21, wherein the polymer dissolved in the solvent for forming an alignment film is polyamic acid which is a precursor of polyimide.

23. The method of manufacturing a liquid crystal display according to claim 21, wherein the changing of the chemical structure of the variable compound is performed by irradiating the light.

24. The method of manufacturing a liquid crystal display according to claim 21, wherein the removing of the solvent having the changed chemical structure is performed by heating and evaporating the solvent having the changed chemical structure.

25. The method of manufacturing a liquid crystal display according to claim 24, wherein a temperature for heating the solvent having the changed chemical structure is room temperature or more and 230° C. or less.

26. The method of manufacturing a liquid crystal display according to claim 21, wherein the removing of the solvent having the changed chemical structure is performed by depressurizing and evaporating the solvent having the changed chemical structure.

27. The method of manufacturing a liquid crystal display according to claim 26, wherein the depressurizing is performed under 1 mmTorr or less to be evaporated.

28. The method of manufacturing a liquid crystal display according to claim 21, wherein the alignment film has a regulatory force in which the liquid crystal is aligned in a film plane by an alignment regulatory force of the liquid crystal.

29. The method of manufacturing a liquid crystal display according to claim 28, wherein the liquid crystal display is an IPS mode liquid crystal display.

30. The method of manufacturing a liquid crystal display according to claim 21, wherein the alignment film has a regulatory force in which the liquid crystal is aligned in a standing direction of 30 degrees or more from a film plane by an alignment regulatory force of the liquid crystal.
